(19) United States
(12) Patent Application Publication
(10) Pub. No.: US 2021/0038203 A1
(43) Pub. Date: Feb. 11, 2021

(54) NEEDLE TIPS, NEEDLES FOR TISSUE COLLECTION, METHODS OF FABRICATION, AND METHODS OF USE

(71) Applicant: HOYA CORPORATION, Tokyo (JP)

(72) Inventors: Robb Morse GAVALIS, Westborough, MA (US); Christopher KADAMUS, West Roxbury, MA (US)

(73) Assignee: HOYA CORPORATION, Tokyo (JP)

(21) Appl. No.: 16/968,540

(22) PCT Filed: Feb. 8, 2019
(86) PCT No.: PCT/US2019/017369

§ 371 Date: Aug. 7, 2020

Related U.S. Application Data
(60) Provisional application No. 62/628,856, filed on Feb. 9, 2018.

(51) Int. Cl. A61B 10/02; A61B 10/04

(52) U.S. Cl. CPC A61B 10/0266; A61B 2010/045; A61B 10/04

(57) ABSTRACT

Embodiments of the present disclosure include devices, systems, and methods for acquiring a tissue sample in an endoscopic procedure. In one implementation, a biopsy needle is provided. The biopsy needle includes an elongated body extending along a longitudinal axis. The elongated body includes a lumen extending therethrough and a distal end comprising at least two tines. A heel between adjacent tines comprises an intersection of two cutting surfaces, each comprising a cutting edge having an inclination angle of at least thirty degrees at each point between a first end of the cutting edge and a second end of the cutting edge. For each of the two cutting surfaces, an angle between the cutting surface and an inner surface of the elongated body is not greater than forty-five degrees in a plane that includes the longitudinal axis.

### BACKGROUND

### Field

The present disclosure generally relates to surgical devices, methods of fabrication of surgical devices, and methods of use of surgical devices. More particularly, and without limitation, the disclosed embodiments relate to devices, systems, and methods for endoscopic tissue collection.

### Background

Fine needle biopsy (FNB) and fine needle aspiration (FNA) are commonly employed during endoscopic ultrasound (EUS) procedures to acquire tissue samples that would have been collected through open surgical or percutaneous techniques in the past. For example, endoscopic ultrasound-guided fine needle aspiration (EUS-FNA) or ultrasound-guided fine needle biopsy (EUS-FNB) has become an effective and minimally invasive diagnostic sampling method in patients with gastrointestinal or pancreatic lesions. EUS-FNA and EUS-FNB combine endoscopic visualization with ultrasound imaging and a sampling device. This allows physicians to use traditional endoscopic visualization to guide their way through the gastrointestinal tract and use ultrasound imaging to provide images of organs and structures beyond the wall of the tract to guide sampling of a desired location. Then, an elongated biopsy needle device is passed through the biopsy channel of the endoscope and is visualized ultrasonically as it penetrates to the desired sampling location to collect a tissue or biological liquid sample.

### SUMMARY

According to an exemplary embodiment of the present disclosure, a biopsy needle is described. The biopsy needle includes an elongated body extending along a longitudinal axis. The elongated body includes a lumen extending therethrough and a distal end comprising at least two tines. A heel between adjacent ones of the at least two tines comprises an intersection of two cutting surfaces, wherein each of the two cutting surfaces comprises a cutting edge having an inclination angle of at least thirty degrees at each point between a first end of the cutting edge and a second end of the cutting edge. For each of the two cutting surfaces, an angle between the cutting surface and an inner surface of the elongated body is not greater than forty-five degrees in a plane that includes the longitudinal axis.

Other disclosed embodiments, and additional features and potential advantages of disclosed embodiments, will be set forth in part in the description that follows, and in part will be obvious from the description, or may be learned by practice of the disclosed embodiments. It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain the principles of the disclosed embodiments as set forth in the accompanying claims.

### BRIEF DESCRIPTION OF THE DRAWINGS

FIGS. 1A-1C show partial perspective views of a lancet tip, a Franseen tip, and an offset tine tip, respectively.

FIG. 2A shows a method of using a grinding plane to form a cutting surface of a needle tip.

FIG. 2B shows a method of using a grinding wheel to form a cutting surface of a needle tip.

FIGS. 3A and 3B show two examples of inclination angle of a cutting edge.

FIG. 4A is a partial perspective view of a biopsy needle 200.

FIG. 4B is another partial perspective view of biopsy needle 200.

FIG. 5A is a partial front view of a distal end 120 of a biopsy needle 100.

FIG. 5B is a partial front view of an implementation 201 of biopsy needle 200.

FIG. 5C is a partial front view of an implementation 202 of biopsy needle 200.

FIG. 5D is a partial front view of an implementation 203 of biopsy needle 200.

FIG. 6A is a partial perspective view of an implementation 204 of biopsy needle 200.

FIG. 6B is a partial perspective view of an implementation 205 of biopsy needle 200.

FIG. 7A is a partial front view of a biopsy needle 300.

FIGS. 7B and 7C show the two bevel planes that form the cutting faces 360 visible in FIG. 7A.

FIGS. 8A and 8B show the directions and rotations of the bevel planes in FIGS. 7A and 7B, respectively.

FIG. 9A shows a partial perspective view of distal end 420 of needle 400.

FIGS. 9B and 9C show two different partial front views of distal end 420 of needle 400.

FIG. 9D shows a cross-section through one wall of needle 400.

FIG. 10A shows a partial perspective view of distal end 421 of needle 401.

FIGS. 10B and 10C show two different partial front views of distal end 421 of needle 401.

FIG. 10D shows a cross-section through one wall of needle 401.

FIG. 11A shows a partial perspective view of distal end 520 of needle 500.

FIG. 11B shows a cross-section of distal end 520 of needle 500 along the bisecting plane indicated in FIG. 11A.

FIG. 12A shows a partial perspective top view of distal end 520 of needle 500 along the longitudinal axis.

FIG. 12B shows a partial front view of distal end 520 of needle 500.

FIG. 13A shows a partial perspective view of distal end 521 of needle 501.

FIG. 13B shows a cross-section of distal end 521 of needle 501 along the bisecting plane indicated in FIG. 13A.

FIG. 14A shows a partial perspective top view of distal end 521 of needle 501 along the longitudinal axis.

FIG. 14B shows a partial front view of distal end 521 of needle 501.

FIG. 15A is a graphical illustration for an exemplary distribution of cutting force along the distal end of biopsy needle 204 of FIG. 6A.

FIG. 15B is a graphical illustration for an exemplary distribution of cutting force along the distal end of biopsy needle 205 of FIG. 6B.

FIG. 15C is a graphical illustration for an exemplary distribution of cutting force along the distal end of biopsy needle 200 of FIG. 4A.

FIGS. 16-19 show degrees of freedom in a multi-axis laser system for cutting needle geometry.

FIGS. 20A and 20B show needles having echogenic markings.

FIG. 21 shows examples of echogenic markings made with several different processes under ultrasound illumination.

FIGS. 22A, 22B, and 23 show examples of needles having echogenic markings.

FIG. 24A shows a photograph of a marking cut with a fiber laser, and FIG. 24B shows a photograph of a marking cut with a femtosecond laser.

FIGS. 25A-25C show examples of alternative marking shapes.

FIG. 26 shows an example of diamond marking spacing in a 19G needle.

FIG. 27 shows an example of diamond marking spacing in a 22G needle.

FIG. 28 shows an example of diamond marking spacing in a 25G needle.

FIG. 29A-C show cross-sections of examples of echogenic markings.

### DETAILED DESCRIPTION

The embodiments of the present disclosure include devices (e.g., biopsy needles), systems, and methods for acquiring a tissue sample in an endoscopic procedure. Advantageously, the exemplary embodiments may allow for the acquisition of adequate tissue or biological liquid samples in EUS-FNA or EUS-FNB procedures with high success rates, thereby improving the efficiency and effectiveness of these endoscopic procedures.

The disclosed embodiments include devices, systems, and methods for collecting an adequate tissue sample in an endoscopic procedure. Embodiments of the present disclosure can be implemented in an endoscopic system for collecting tissue samples at desired locations in or in proximity of the gastrointestinal or pancreatic tract, where soft tissue samples are typically collected for diagnostic biopsy. Advantageously, embodiments of the present disclosure allow for effective collection of a desired amount of tissue sample at a desired location, thereby increasing the success rate and efficiency of collecting adequate tissue samples in an endoscopic procedure.

As described herein, an endoscope, such as an ultrasound endoscope, typically includes a proximal end, a distal end, and an internal working channel extending between the distal end and the proximal end. A proximal end may refer to a point or a location along the length of the endoscope closer to a physician or a medical practitioner. A distal end may refer to a point or location along the length of the endoscope closer to a sampling location in the body of a patient. A biopsy needle device is typically introduced into the working channel of the endoscope from the proximal end to the distal end of the endoscope until a distal end of the needle device approximates or reaches a desired location for collecting one or more tissue samples.

Reference will now be made in detail to embodiments and aspects of the present disclosure, examples of which are illustrated in the accompanying drawings. Where possible, the same reference numbers will be used throughout the drawings to refer to the same or like parts.

One of the main considerations to optimize EUS-FNA or EUS-FNB procedures is to acquire an adequate tissue sample with a minimal number of passes and a minimal risk of injury to surrounding tissue in the patient. Despite the widespread usage of EUS-FNA and EUS-FNB techniques, one limitation of these techniques is that they often provide tissue samples with scant cellularity and/or lack of histologic architecture. This inadequacy of the collected tissue samples limits the performance of histologic grading of malignant tissues and/or subsequent molecular biological analysis.

Endoscopic FNA and FNB needles tend to have a lower tissue collection success rate than do more traditional percutaneous biopsy needles. This low rate is primarily due to the small needle gauge, the difficulty getting the endoscopic needle to the correct tissue to be collected and the needle tip geometry. Depending on the reporting physician, diagnostically adequate tissue can be collected at a rate of 50% to 90%. However, variation in patient population and skill of the physician can skew these success rates considerably. Failure to collect a sufficient amount of the desired tissue can mean that the patient has to be under anesthesia longer, more devices may be used and in the worst cases, patients require additional procedures to obtain sufficient tissue. The goal of designing an improved method of tissue collection would be to increase both the rate of collection of diagnostically adequate tissue as well as to reduce the amount of fanning passes and/or the number of times a device must be inserted in the endoscope to acquire adequate tissue. Such reductions could not only reduce the number of needle sticks in the patient but could also reduce the time required for the procedure (as well help eliminate a need for repeat procedures).

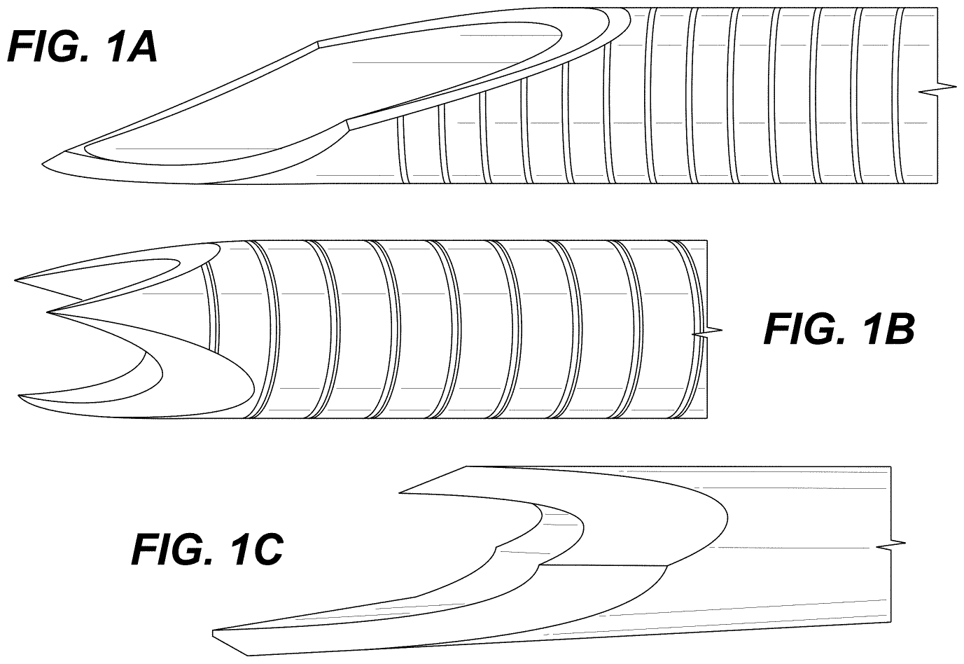

Existing endoscopic needles are not optimized to overcome the limitations of EUS-FNA or EUS-FNB procedures discussed above. For example, the majority of existing endoscopic FNA or FNB needles have a needle tip geometry similar to that of a traditional lancet needle (e.g., a tip as shown in FIG. 1A, which has three grind planes). Lancet needles were designed in the early twentieth century and have been commonly used for injection or percutaneous puncture. Thus, lancet needles are designed to cut and split tissue with the lowest penetration force, apply the least drag to reduce pain, and prevent tissue from entering the needle inner lumen. These design criteria are contrary to the tissue collection purpose of an endoscopic FNA or FNB needle. The use of lancet-style needle tip geometry in endoscopic FNA or FNB needles contributes to highly variable success rates of collecting tissue samples.

Other endoscopic FNA or FNB needles use alternative needle tip geometries, such as back bevel needles, Franseen needles (e.g., a tip as shown in FIG. 1B, which has three grind planes), and offset tine needles (e.g., a tip as shown in FIG. 1C, which has four grind planes). However, the acquisition rate of adequate amount of tissue could still be improved. Obstacles include the small gauge of endoscopic FNA or FNB needles, the difficulty in getting an endoscopic needle to the correct sampling location, and the needle tip geometry. Depending on the reporting physician, adequate tissue samples can be collected at a success rate as low as 50% using existing needles. Variation in patient population and skill of the physician can further vary the success rate considerably. Failure to acquire adequate tissue samples may result in longer anesthetization of the patient, the use of more devices and/or more passes of a device, and even additional procedures to obtain adequate tissue samples for some patients.

Therefore, there is a need for improved biopsy needles or biopsy needle tips that increase the effectiveness of tissue sample collection in EUS-FNA or EUS-FNB procedures, and thus reduce the passes performed by the physician to acquire an adequate tissue sample. Such biopsy needles or biopsy needle tips thus improve the efficiency and success rates of EUS-FNA or EUS-FNB procedures.

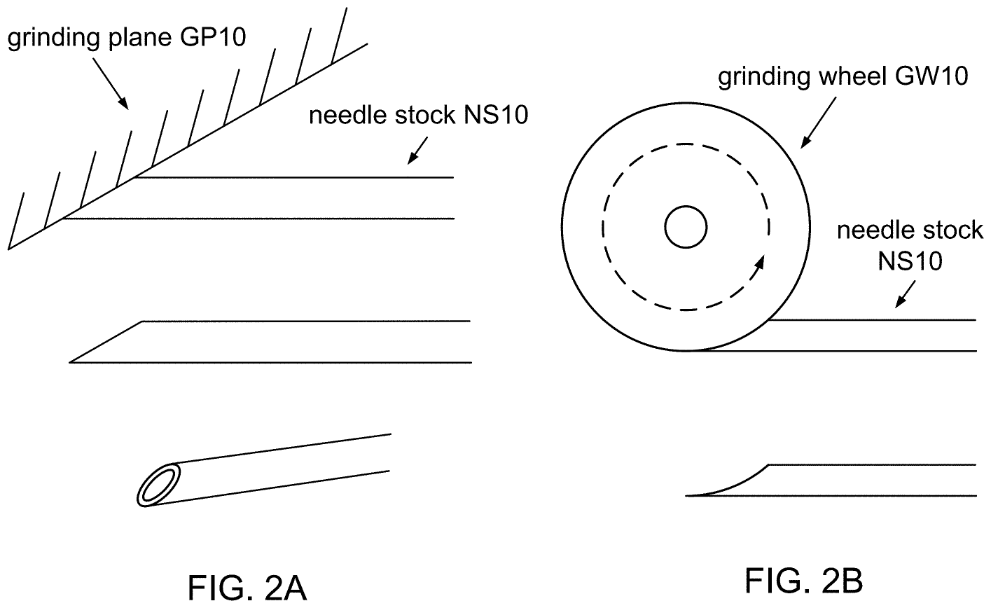

Alternate needle tip geometries have become available in recent years. However, all of these needles are manufactured using the same traditional grinding techniques that have been used to create needles for decades. Even the more exotic existing needle tip geometries (e.g., forked tip, offset tine) are made by angling grinding wheels in different directions. Manufacturing needles by using grinding techniques alone limit the types of geometries that can be created, in part because such techniques are restricted to producing flat or cylindrical cutting surfaces. FIG. 2A shows a conventional method of using a grinding plane GP10 (e.g., the flat surface of a grinding wheel) to form a bevel plane of a needle tip into a piece of needle stock NS10 (with the beveled tip also shown in profile and in a partial perspective view). FIG. 2B shows a conventional method of using a grinding wheel GW10 to form a cylindrical surface of a needle tip into a piece of needle stock NS10 (with the formed tip also shown in profile).

Several versions of novel needle tip geometries are shown in this disclosure. Most of the particular versions shown have a common base geometry: each needle contains four sets of tines that are 90 degrees from each other, spread evenly around the longitudinal axis of the tube. Needles having the same features as a described four-tine embodiment (e.g., needle 200, 201, 202, 203, 300, 400, 401, 500, or 501) but fewer (e.g., three) or more (e.g., five or six) tines are also enabled, expressly contemplated, and hereby disclosed.

The term “tip of a tine” is used herein to indicate the most distal point of a tine of a needle. The term “base of a tine” is used herein to indicate the most proximal point of a tine of a needle, which may also be a base of an adjacent tine. The term “heel” is used herein to indicate the region between two adjacent tines of a needle which is near to the base of the two tines. A cutting surface of a needle may also be referred to as a cutting face or needle facet.

Angles which may be considered when optimizing cutting geometry include the inclination angle, the rake angle, and the bevel angle, and it has been noted in the art that such angles define a geometry of the cutting edge. The inclination angle is the angle between the tangent at a given point of a cutting edge (e.g., the line along a straight cutting edge) and the plane perpendicular to the longitudinal axis of the needle at that point, as measured in a plane parallel to the longitudinal axis of the needle. The rake angle is the angle of the cutting face relative to the work surface (in this case, the tissue) at the cutting point, as measured in a plane with a normal vector that is tangent to the cutting edge (i.e., a plane that is orthogonal to the tangent to the cutting edge). The bevel angle may be defined as the angle of the cutting surface relative to the longitudinal axis of the needle, as measured in a plane that includes the longitudinal axis and a vector normal to the cutting surface. Factors that guide the selection of a bevel angle of a biopsy needle may include, for example, any of the following: the hardness or softness of the tissue to be sampled, the gauge of the biopsy needle, the number of tines in the needle tip, the geometry of the tines, etc.

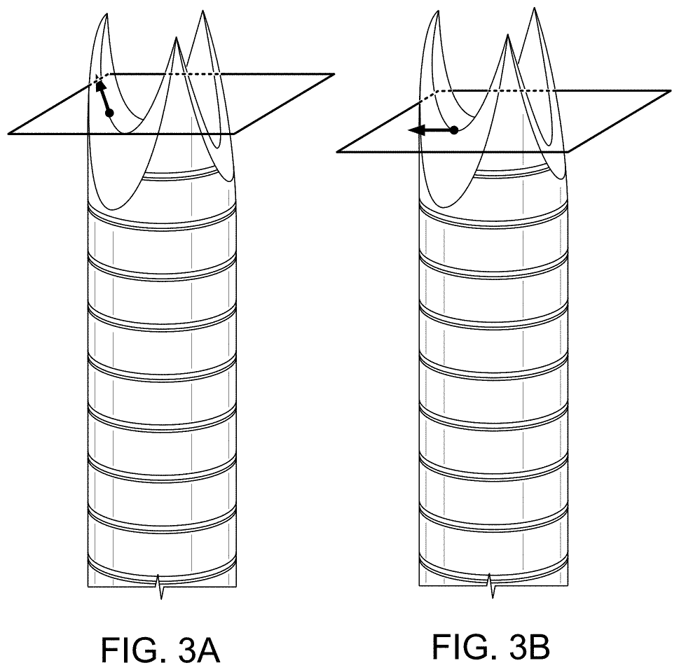

FIGS. 3A and 3B show two examples of inclination angle along a cutting edge of the Franseen needle shown in FIG. 1B. At a needle tip (e.g., the tip of a tine), the inclination angle is effectively 90 degrees, and thus its cutting ability is optimized. The inclination angle decreases at intermediate points along the cutting edge, as shown in FIG. 3A. At a ground needle bevel heel (e.g., as shown in FIG. 3B), the inclination angle is effectively 0 degrees or parallel to the tissue. At the exact point of the bevel heel, therefore, the needle is not cutting at all, but rather pushing the tissue. Since no cutting occurs, the needle tends to push the tissue aside rather than guiding it into the inner diameter of the needle. The shallower the bevel and wider the grind, the more points there are where the inclination angle is close to zero. As such, cutting is poor and so are the biopsy results. Experiments have shown that inclination angle affects the forces on the needle upon insertion and that a needle having an inclination angle that is too low fails to successfully cut the tissue.

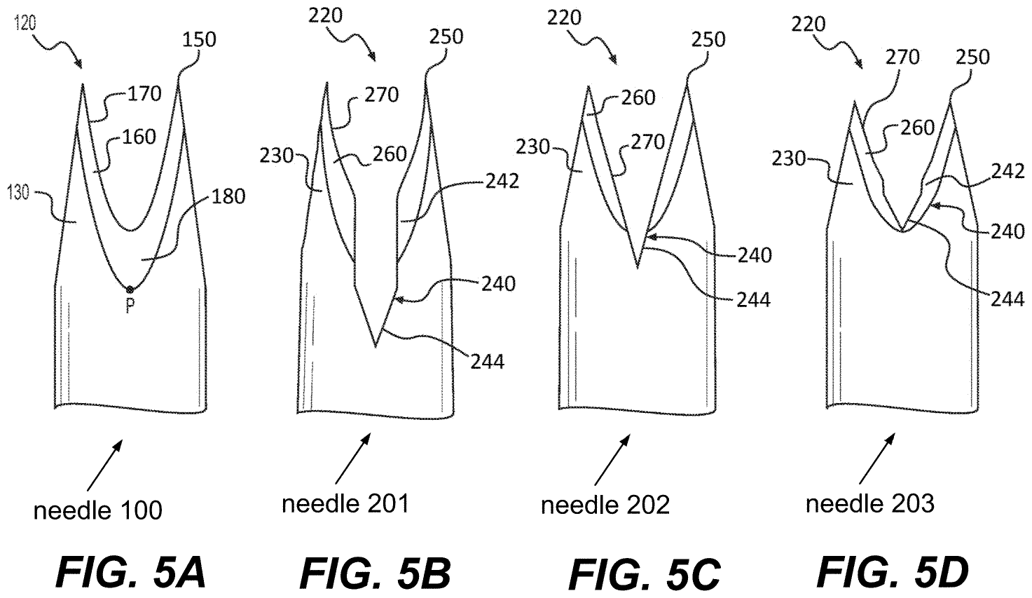

FIG. 5A is a partial front view of distal end 120 of a biopsy needle 100 having four tines 130 (two are seen in this view) that are formed by four cutting surfaces 160 (one is seen in this view), each formed in a corresponding bevel plane (e.g., ground, for example as shown in FIG. 2A). In this example, tines 130 are radially evenly spaced apart, as are the bevel planes in which cutting surfaces 160 are formed. Each cutting surface 160 is shared by two tines 130, and each tine 130 has a tine tip 150 at which the two cutting surfaces that form the tine meet. Each cutting surface includes a cutting edge 170 and a bevel heel 180. As shown in FIG. 5A, bevel heel 180 resides within a lowest point “P” of the corresponding cutting surface 160 along the longitudinal axis of biopsy needle 100.

The inclination angle of cutting edge 170 of needle 100 is at its highest value at tine tip 150 and decreases towards the bottom of cutting surface 160. The inclination angle is close to or about zero at bevel heel 180 of cutting surface 160. Locations along cutting edge 170 that have lower inclination angles, such as bevel heel 180, result in higher cutting forces applied to the tissue during needle insertion, which in turn causes the tissue to be pushed away or around the needle rather than being cut and entering the lumen of the needle. Therefore, distal end 120 of needle 100 has poor cutting performance at and near bevel heels 180 of its cutting surfaces 160.

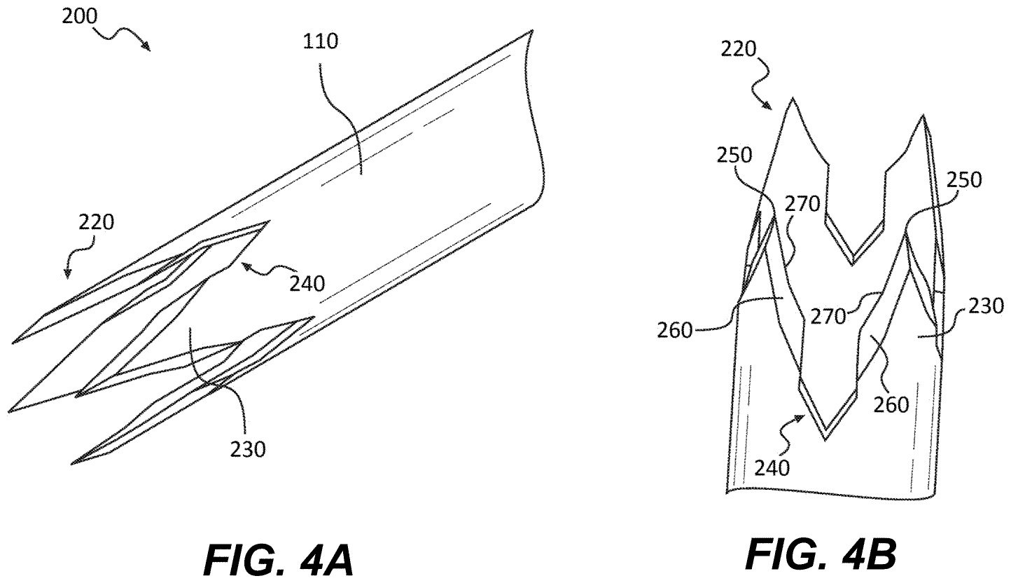

FIG. 4A shows a partial perspective view of a biopsy needle 200 in which each tine 230 is cut at the tip to form wedge pattern “bevels” on each side of the tine that sharpen the needle (as described herein, these “bevels” could be planar or have a variable (curved) surface geometry). After the front “bevels” (between the tines), a laser or other method as disclosed herein creates a “V” shaped or other specified cut. In some embodiments, this cut is orthogonal to the longitudinal axis of the needle, in other embodiments the cut angles are shallower than 90 degrees in both single and/or variable angle configurations.

One approach to increasing the average inclination angle of such a cutting edge is to remove a portion of the bevel heel which has the lowest inclination angles. FIGS. 4A and 4B are partial perspective views of an exemplary biopsy needle 200. As shown in FIGS. 4A and 4B, biopsy needle 200 includes an elongated body 110 extending along a longitudinal axis. Elongated body 110 includes a distal end 220 and a lumen extending therethrough. As described herein, distal end 120 may also be referred to as the biopsy needle tip. Distal end 120 has a plurality of tines 230 and a plurality of cutouts 240. Tines 230 may each have a symmetric shape and be formed by two cutting surfaces. For example, as shown in FIG. 4B, tines 230 may each have two cutting surfaces 260 formed in bevel planes that meet at a tine tip 250. Each cutting surface 260 has a cutting edge 270. Every point along cutting edge 270 of cutting surface 260 has an inclination angle.

As noted above, the inclination angle is the angle between the tangent at a given point of a cutting edge of a needle and the plane perpendicular to the direction of needle insertion. An overall average inclination angle of a needle is an average of the inclination angles at a set of the points along the cutting edge of the needle. (In practice, between one thousand and ten thousand points, for example, may be used to calculate an average inclination angle.) For example, as shown in FIG. 4B, the inclination angle of cutting edge 270 is at its highest value at tine tip 250 and decreases towards the points at lower portions of cutting surface 260. An overall average inclination angle of biopsy needle 200 is the average inclination angle of all points or locations of all the cutting edges 270 of distal end 220.

As described herein, the inclination angle affects the cutting force applied to the tissue at a given point of a cutting edge. Increasing the inclination angle of a cutting edge of a biopsy needle reduces the cutting forces applied to the tissue being cut, which in turn leads to more efficient tissue cutting and better tissue samples for biopsy (e.g., longer tissue sample or tissue sample with sufficient cellularity and/or histologic architecture).

Advantageously, to increase the inclination angle of cutting edge 270 and/or the overall average inclination angle of the needle, distal end 220 of biopsy needle 200 includes a plurality of cutouts 240 residing at locations that correspond to low inclination angles in needle 100 as shown in FIG. 5A. For example, cutouts 240 may reside at the locations of bevel heels 180 of ground bevels 160 of FIG. 5A. FIGS. 5B-5D show implementations 201, 202 and 203, respectively of needle 200 having cutouts 240 that may each extend into or through a bevel heel, and thus separate a ground cutting surface into two secondary cutting surfaces, thereby eliminating bevel heels. Each cutting surface 260 then has a separate cutting edge 270. Advantageously, cutouts 240 eliminate or replace locations of the cutting edges that have low inclination angles, thereby increasing the overall average inclination angle of distal end 220 and thus the cutting performance of biopsy needle 200. For example, an overall average inclination angle of distal end 220 may range from about 50° to about 85°.

Cutouts 240 can be formed by any suitable micro-machining operation or method, which may include any of laser cutting, electrical discharge machining (EDM) cutting, water-jet cutting, and chemical etching. Cutouts 240 can have different configurations. In some exemplary embodiments (e.g., needle 201 as shown in FIG. 5B), each cutout 240 may include a longitudinally straight section 242 and a V-shaped section 244, both of which cut through or extend beyond the bevel heel of a ground cutting surface. In such instances, both longitudinally straight section 242 and V-shaped section 244 extend beyond point “P” (as indicated in FIG. 5A) along the longitudinal axis of the biopsy needle. As described herein, point “P” is the lowest point of the grind plane of cutting surface 160 as indicated in FIG. 5A or, analogously, the lowest point of a grind plane of cutting surface 260 along the longitudinal axis of biopsy needle 200.

The inclination angle of longitudinally straight section 242 is about 90°, greater than the inclination angles of the locations along cutting edge 270 that are replaced by longitudinally straight section 242. Additionally, the inclination angle of V-shaped section 244 is greater than the inclination angles of locations at or around the bevel heel that are replaced by V-shaped section 244. Therefore, by eliminating or replacing locations of distal end 120 that have low inclination angles, cutouts 240 advantageously increase the overall average inclination angle of distal end 220, thereby reducing cutting forces applied to the tissue and improving the cutting performance relative to that of biopsy needle 100.

In other exemplary embodiments (e.g., needle 202 as shown in FIG. 5C), each cutout 240 may include only V-shaped section 244, which cuts through or extends beyond the bevel heel of a ground bevel surface. In such instances, V-shaped section 244 extends beyond point “P” along the longitudinal axis of the biopsy needle. Alternatively (e.g., as in needle 203 as shown in FIG. 5D), cutout 240 may include longitudinally straight section 242 and V-shaped section 244, both of which cut into or reside within a bevel heel of a ground bevel surface. In such instances, both longitudinally straight section 242 and/or V-shaped section 244 of cutout 240 reside within point “P” along the longitudinal axis of the biopsy needle.

In further exemplary embodiments, longitudinally straight section 242 of cutout 240 may at least partially reside within a bevel heel of ground bevel surface while V-shaped section 244 may extend beyond the bevel heel (not shown). Alternatively, longitudinally straight section 242 of cutout 240 may reside within a bevel heel of a ground bevel surface while V-shaped section 244 may partially reside within and partially extend beyond the bevel heel (not shown).

As described herein, the widths and/or lengths of longitudinally straight section 242 and V-shaped section 244 of cutout 240 may be predetermined such that locations along a ground bevel surface are eliminated, including a bevel heel. In some embodiments, the biopsy needle may have a hypodermic gauge ranging from about 27 G to about 17 G, or an outer circumference ranging from about 1.294 mm to about 4.628 mm, respectively. The needle may be made from hypodermic tubing made of, for example, stainless steel, a cobalt-chromium alloy, or Nitinol (nickel titanium alloy). In such instances, the sum arc lengths of cutouts 240 are less than about 75% and more than about 10% of the total outer circumference of the biopsy needle. As described herein, an arc length of cutout 240 is the length of cutout 240 extending along the outer circumference of the biopsy needle. Increasing the total arc lengths of cutouts 240 increases the overall average inclination angle of the biopsy needle and reduces the cutting forces applied to the tissue by the biopsy needle.

For an exemplary biopsy needle having a hypodermic gauge ranging from about 17 G to about 27 G, the longitudinal lengths of cutouts 240 along the length of the biopsy needle may extend to the proximal edge of the bevel heel (or point “P” as shown in FIG. 5A) at the minimum. Alternatively, the longitudinal lengths of cutouts 240 may extend beyond the proximal edge of the bevel heel (or point “P” as shown in FIG. 5A). In such instances, the longitudinal lengths of cutouts 240 are less than about 15 mm, for example. Increasing the longitudinal lengths of cutouts 240 may increase a degree of radial deflection of the tines during needle insertion and/or tissue collection.

Additionally, the widths and/or lengths of longitudinally straight section 242 and V-shaped section 244 of cutout 240 may be predetermined such that the integrity of the tines can be maintained during the insertion and/or extraction of the biopsy needle. As described herein, the integrity of the tines may be maintained when the tines are wide enough to have sufficient strength to avoid from being bent, deformed, or damaged due to frictional and/or reactive forces from the tissue applied to the tines during needle insertion or extraction. Such bending, deformation, or damage of the tines may further impede the tissue from entering the lumen of the biopsy needle for collection, and/or may result in inadvertent damage to the tissue at the sampling location. Therefore, the widths and/or lengths of cutouts 240 may be predetermined based on the widths of the tines and/or the size of the ground bevel surface.

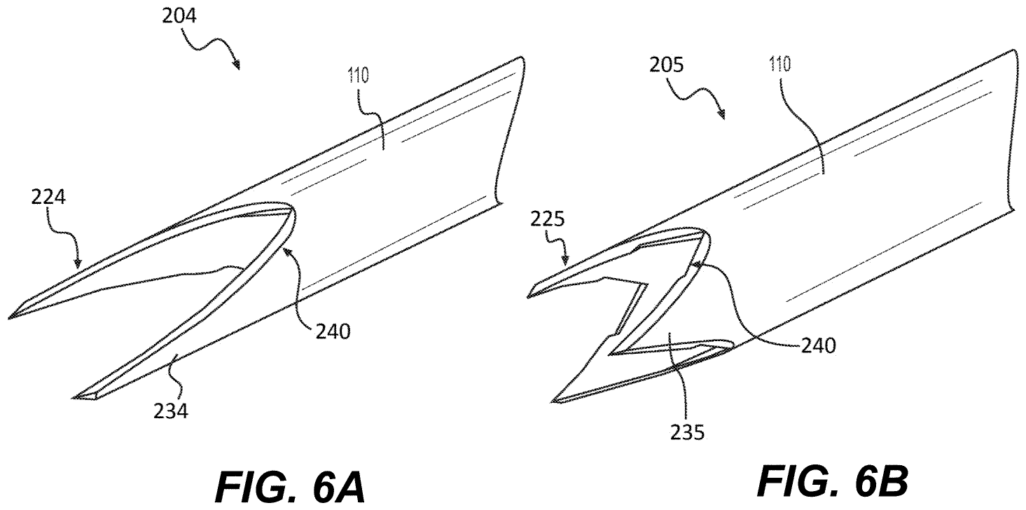

A biopsy needle may have a predetermined number of tines formed on a corresponding number of grind planes suitable for a desired endoscopic biopsy procedure. For example, biopsy needle 200 may be implemented to include two tines 234 formed by two grind planes as shown in FIG. 6A (two-plane biopsy needle 204) or three tines 235 formed by three grind planes as shown in FIG. 6B (three-plane biopsy needle 205). In such instances, the distal end of the biopsy needle includes a same number of cutouts 240 located between each set of adjacent tines. In some embodiments, distal end 220 of biopsy needle 200 includes four tines 230 formed by four grind planes (four-plane biopsy needle 200). Four tines 230 of a four-plane biopsy needle 200 are evenly radially spaced apart in rotational symmetry as well as in plane symmetry about two orthogonal longitudinal planes that are parallel to the longitudinal axis of biopsy needle 200. For example, as shown in FIGS. 4A and 4B, distal end 220 includes four tines 230 formed by four grind planes. Distal end 220 further includes eight cutting surfaces 260 and eight cutting edges 270. Each of the four grind planes is oblique to the longitudinal axis of biopsy needle 200 and orthogonal to two neighboring grind planes in an x-y plane perpendicular to the longitudinal axis. Each of the four grind planes is oblique to the longitudinal axis at a desired bevel angle ranging from about 5° to about 20°. In some embodiments, the four grind planes are oblique to the longitudinal axis at the same desired bevel angle.

As described herein, the bevel angle of the grind planes of biopsy needle 200 may be selected based on various factors, such as the hardness or softness of the tissue to be sampled, the gauge of biopsy needle 200, the number of tines 230, the geometry of tines 230, etc. Decreasing the bevel angles of the grind planes increases the inclination angles of cutting edges 270, and thus increases the overall average inclination angle of biopsy needle 200. This increase in turn reduces the cutting forces applied to the tissue by distal end 220 of biopsy needle 200, allowing for more efficient tissue cutting and better tissue samples for biopsy. Exemplary ranges of bevel angles for the needle tip embodiments disclosed herein include any of the following ranges: from five (or seven, or ten, or twelve, or fifteen, or 20) degrees to 25 (or 30, or 35, or 40, or 45) degrees.

Using a “V” cut to remove the area of the needle bevel heel that has low inclination angles creates a steeper cutting geometry where the inclination angles are high. Because the point that forms the bottom of the “V” cut is the only point at which the inclination angle is at or close to zero, the overall average inclination angle of the needle is larger when compared to the same four-plane needle without such “V” cutouts. The needle with the “V” cutouts will thus cut tissue with less force and provide better tissue biopsy.

The vertical walls (parallel to the longitudinal axis of the needle) cut into the heel of the bevel create a steep inclination angle, and the sharp angled V-shaped cut proximal to the vertical walls minimize the area of low inclination angle. These features improve the overall average inclination angle of the needle and provide a needle that cuts more efficiently. Other examples (e.g., needle 202 as shown in FIG. 5C) include “V” cutouts but do not include such vertical walls.

It is noted that making the cutout too long (i.e., extending too far toward the proximal end of the needle) could result in the flexing of the tines either inwardly or outwardly when the needle is inserted into tissue. Making the cutout too wide may weaken the tines to the point at which they may bend and permanently deform. It may be desirable that at its maximum width, the cutout is equal to the tine width, but it may be preferable for the maximum width of the cutout to be smaller than the tine width.

The tips of needles 200, 201, 202, 203, 204, and 205 as shown in FIGS. 4A, 4B, 5B-5D, 6A, and 6B can be made by fully laser-cutting the tip, or by grinding the four tines and using a laser or other method to cut out the heels. The cutout 240 can be made, for example, by using a laser to cut the material from the needle or a plunge EDM head to eliminate the material.

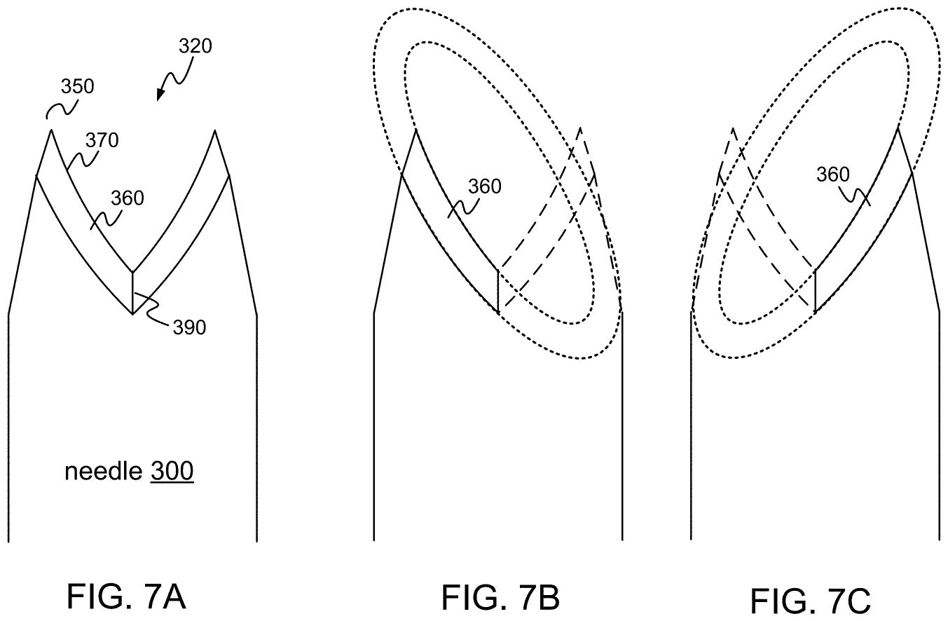

As described above with reference to needle 200, an overall average inclination angle of a cutting edge may be increased by cutting out or otherwise modifying a portion of a bevel heel in a ground bevel plane. An additional or alternative approach to increasing an overall average inclination angle of a needle tip is to form adjacent sides of adjacent tines of the needle tip by using bevel planes that intersect the needle. FIG. 7A shows a partial front view of one example of a needle tip 300 in which the bevel planes of adjacent sides of adjacent tines intersect to form cutting faces whose cutting edges have a non-zero inclination angle all along their respective lengths. The dotted ellipses in FIGS. 7B and 7C show the two bevel planes that form the cutting faces visible in FIG. 7A, and the dashed lines in these figures show a portion of the needle tip that each of these bevel planes intersects.

As compared to a ground bevel plane that forms adjacent sides of adjacent tines of a needle (e.g., as in the Franseen needle shown in FIG. 1B), the intersection of bevel planes between adjacent tines of needle 300 as shown in FIG. 7A excludes the area of low inclination angles from the heel and creates a steeper cutting geometry such that the inclination angle remains high. Because the point at which the cutting edges intersect is the only point at which the inclination angle is at or close to zero, the overall average inclination angle of the needle tip is larger as compared to a needle tip in which adjacent sides of adjacent tines are formed by a common bevel plane, and the needle has a nearly continuous cutting bevel. The needle tip having such geometry will thus cut tissue more smoothly and efficiently and with less force, providing better tissue biopsy performance.

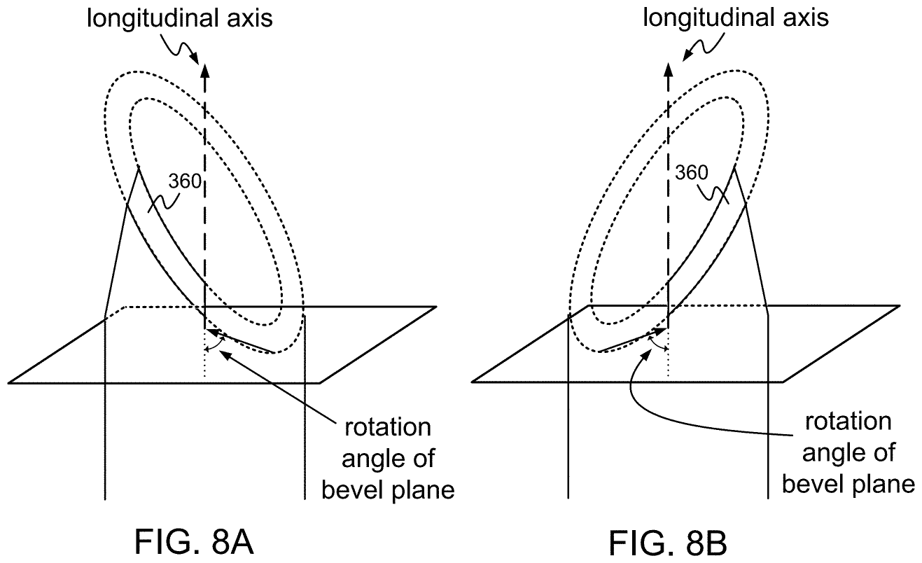

The direction of a bevel plane may be characterized as the direction of the projection, onto a plane that is orthogonal to the longitudinal axis of the needle, of a vector that is normal to the bevel plane and intersects the longitudinal axis of the needle. The direction of the bevel plane shown in FIG. 5A, for example, is directly into the paper and is 45 degrees (relative to the longitudinal axis of the needle) from each of the tines shown. Taking this direction as a reference, it may be seen in FIG. 8A that the left-side cutting face of needle 300 is formed by a bevel plane that is rotated around the longitudinal axis of the needle to the right, and in FIG. 8B that the right-side cutting face of needle 300 is formed by a bevel plane that is rotated around the longitudinal axis of the needle similarly to the left.

It may be understood that rotating a bevel plane in a direction away from its tine (e.g., as shown in FIGS. 7B and 8A and also in FIGS. 7C and 8B) causes the height of the tine at the outer wall of the needle to rise, which in turn causes the inclination angle of the cutting edge that extends between the inner and outer walls of the needle at the tip of the tine to decrease. For a case in which the directions of the bevel planes that form each side of a tine are 90 degrees from the tine (and therefore 180 degrees from each other), this cutting edge becomes horizontal and has an inclination angle of zero. For such reasons, it may be desirable for the direction of each bevel plane to be not more than 50, 55, 60, 65, 70, or 75 degrees (relative to the longitudinal axis of the needle) away from the tip of a tine that is formed in part by the bevel plane. Alternatively or additionally, it may be desirable to cut away the outer corner of each tine to replace this cutting edge with two cutting edges that have higher inclination angles (e.g., as shown in FIG. 6A).

The dimensions and/or shapes of the V-shaped section and/or the longitudinally straight section of the cutouts of the biopsy needle may be predetermined such that the integrity of the tines is maintained during insertion and/or extraction of the biopsy needle and such that the heels of the primary ground bevels are eliminated. As noted above, in some embodiments, biopsy needle 300 may have a hypodermic gauge ranging from about 27 G to about 17 G, or an outer circumference ranging from about 1.294 mm to about 4.628 mm, respectively. Needle 300 may be made from hypodermic tubing made of, for example, stainless steel, a cobalt-chromium alloy, or Nitinol (nickel titanium alloy).

In some embodiments, the lengths of the cutouts along the longitudinal axis are greater than the widths of the tines. Cutouts longer than the widths of the tines may allow the tines to be radially deflectable. For example, as the biopsy needle is inserted into a sampling location, the tines may radially deflect outward relatively to the longitudinal axis. As the biopsy needle is extracted from the sampling location, the tines may radially collapse inward relatively to the longitudinal axis. Advantageously, the radial deflection of the tines allows the tissue cut by the distal end to enter the lumen of the biopsy needle more easily (e.g., with less hindrance) during needle insertion and to retain the tissue within the lumen of the biopsy needle (e.g., with more frictional and/or supporting surface) during needle extraction.

As noted above, making the bevel planes too steep may result in tines that flex either inwardly or outwardly when the needle is inserted into tissue. As also noted above, although the particular embodiment shown in FIG. 7A and discussed above has four tines, additional embodiments could have three tines, or up to five tines or more, instead.

It is not possible to use a flat grinding technique (e.g., as shown in FIG. 2A) or a cylindrical grinding technique (e.g., as shown in FIG. 2B) to form bevel planes such that at least one of the bevel planes intersects the needle. The cutting surfaces 360 may be formed using another suitable micro-machining operation or method, such as laser cutting, electrical discharge machining (EDM) cutting, water jet cutting, or chemical etching.

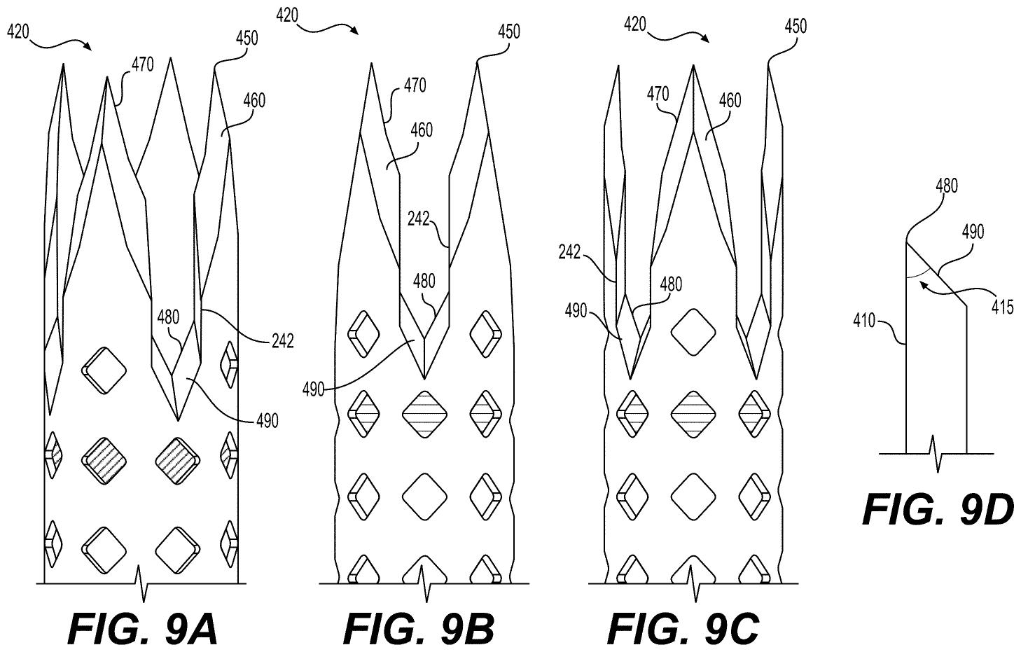

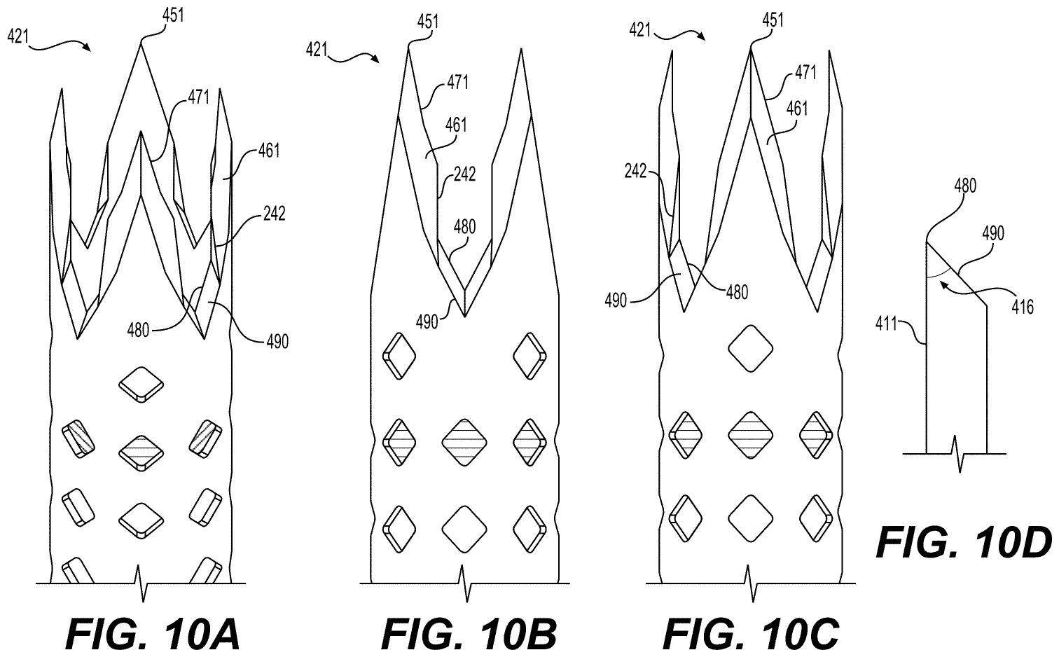

The implementations 400 and 401 of biopsy needle 200 as shown in FIGS. 9A-9C and 10A-10C, respectively, use a similar principle of increasing the amount of cutting surface as needle 200 but change the geometry of the “V” to form an angled V-bevel near the base of the “V” cut. FIG. 9A shows a partial perspective view of distal end 420 of needle 400 (including tips 450, cutting faces 460 and 490, cutting edges 470 and 480, and longitudinally straight section 242 as described above), and FIGS. 9B and 9C show two different partial front views. FIG. 9D shows a cross-section through one wall of needle 400 at one of cutting surfaces 490, showing an angle 415 between cutting surface 490 and an inner wall 410 of the needle. Angle 415 may be selected from any of the following ranges: from 10, 15, 20, 25, or 30 degrees to 60, 65, 70, 75, or 80 degrees (e.g., 20, 25, 30, 35, 40, 45, or 60 degrees).

FIG. 10A shows a partial perspective view of distal end 421 of needle 401 (including tips 451, cutting faces 461 and 490, cutting edges 471 and 480, and longitudinally straight section 242 as described above), and FIGS. 10B and 10C show two different partial front views. FIG. 10D shows a cross-section through one wall of needle 401 at one of cutting surfaces 490, showing an angle 416 between cutting surface 490 and an inner wall 411 of the needle. Angle 415 may be selected from any of the following ranges: from 10, 15, 20, 25, or 30 degrees to 60, 65, 70, 75, or 80 degrees (e.g., 20, 25, 30, 35, 40, 45, or 60 degrees).

Needle 400 as shown in FIGS. 9A-9C has a deeper V cut, while needle 401 as shown in FIGS. 10A-10C has a shallower V cut that is aligned with the base of the bevels. This geometry allows for nearly continuous cutting until the very base of the V by eliminating the full wall thickness geometry of needle 200 as shown in FIGS. 4A-B and by creating high inclination angles, even near the base of the V cut.

In the embodiments shown, the angle of the cut is 45 degrees; however, these cuts could have any angle smaller than 90 degrees, as long as material conditions and physical limitations allow. A steeper angle results in a shorter cut (as measured along a line that is on the cutting surface and is orthogonal to the cutting edge), while a more shallow angle results in a cutting surface that extends farther down the longitudinal axis of the needle. Typically an angle of from 30 to 60 degrees is desirable.

As compared to the ground bevel plane that forms adjacent sides of adjacent tines of the Franseen needle of FIG. 1B, a V-bevel between adjacent tines as shown in FIG. 9A-9C or 10A-10C removes the area of the needle bevel heel that has low inclination angles and creates a steeper cutting geometry where the inclination angle are high. Because the point at which the cutting edges intersect is the only point at which the inclination angle is zero, the overall average inclination angle of the needle is larger when compared to a needle in which adjacent tines share a bevel plane. The needle with the “V” cut outs will thus cut tissue with less force and provide better tissue biopsy.

The vertical walls (parallel to the longitudinal access of the needle) of longitudinally straight section 242 cut into the heel of the bevel create a steep inclination angle, and the sharp angled V-shaped cut proximal to the vertical walls minimizes the area of low inclination angle. These features improve the overall average inclination angle of the needle and provide a needle that cuts more efficiently.

As with the geometries discussed above, making the bevel planes of cutting surfaces 490 too steep may result in tines that flex either inwardly or outwardly when the needle is inserted into tissue, and making the cutout too wide may weaken the tines to the point at which they may bend and permanently deform. It may be desirable that at its maximum width, the cutout is equal to the tine width, but it may be preferable for the maximum width of the cutout to be smaller than the tine width. As noted above, in some embodiments, biopsy needle 400 or 401 may have a hypodermic gauge ranging from about 27 G to about 17 G, or an outer circumference ranging from about 1.294 mm to about 4.628 mm, respectively. Needle 400 or 401 may be made from hypodermic tubing made of, for example, stainless steel, a cobalt-chromium alloy, or Nitinol (nickel titanium alloy).

The tips of needles 400 and 401 as shown in FIGS. 9A-C and 10A-C can be made by fully laser-cutting the tip, or by grinding the four tines and using a laser or other method to cut out the heels. Although the particular embodiments shown in FIGS. 9A-9C and 10A-10C use four grind planes, additional embodiments could have a plurality of planes from two-plane to three-plane, or up to five planes or more, instead. Due to their off-axis nature, the bevels at the base of the V in needles 400 and 401 can only be made using an ultrafast CNC laser or similar process, so long as multiple-axis or 3D ablation techniques are used. The cutout of section 442 with cutting surfaces 490 can be made using a laser to cut the material from the needle, or EDM cutting (e.g. a plunge EDM head) to eliminate the material, or another suitable micro-machining operation or method, such as water jet cutting or chemical etching.

While current grinding methods can produce only flat planes, an ultrafast laser or similar multi-axis technique enables the creation of cutting surfaces that are nonplanar (e.g., curved). In yet another disclosed embodiment, the cutting surfaces of the needle (e.g., any of needles 200, 201, 202, 203, 204, 205, 300, 400, 401) do not remain planar, but instead one or more of the cutting surfaces is curved (e.g., one or more of surfaces 260, 261, 262, 263, 360, 460, 461, and 490). For example, a cutting surface may constitute a curve from the tip of the needle to the start of the V cut. Additionally or alternatively, these bevels could be angularly offset to form needle tips with steeper bevel angles or bevel angles that are not symmetric to the other bevel on the tine. Needles having such configurations may be formed using a multi-axis, CNC cutting system (for example, a multi-axis femtosecond laser).

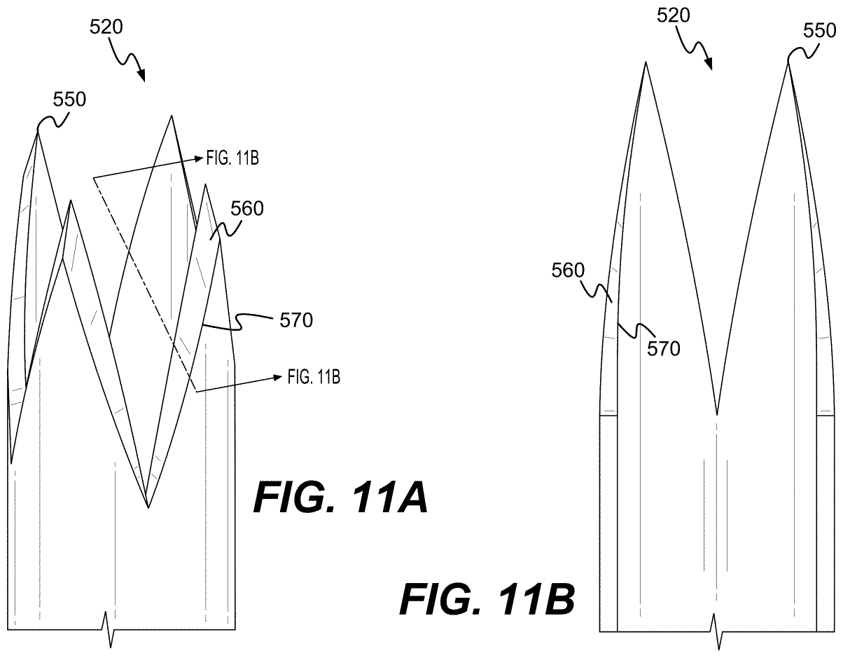

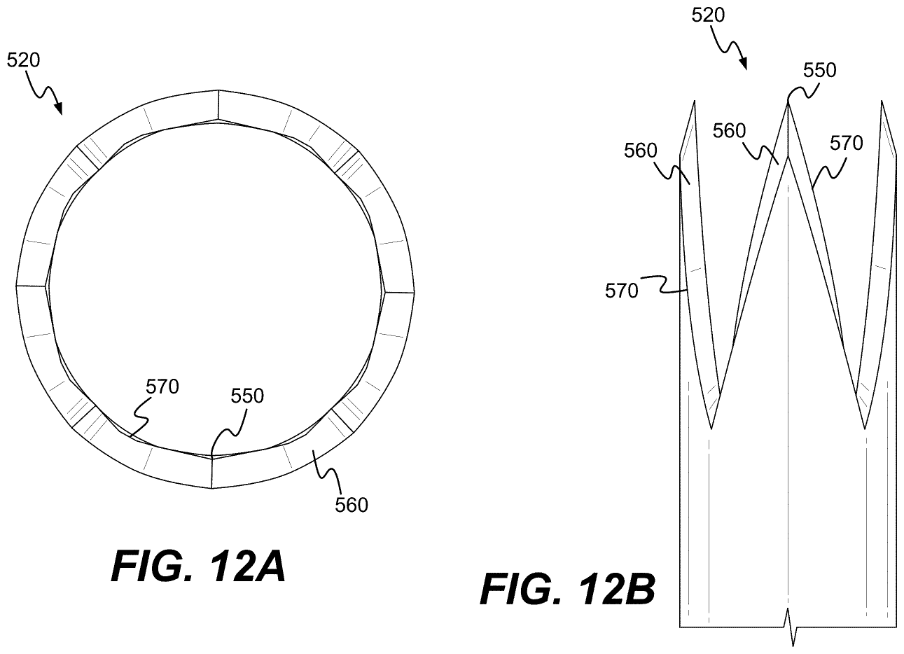

FIGS. 11A, 11B, 12A, and 12B show different views of an embodiment 500 of biopsy needle 200 (including distal end 520, tips 550, cutting surfaces 560, and cutting edges 570) that uses a 3-D curve which starts at the base of the cutting tine and corkscrews slowly until it reaches the vertical point at the base of the “V” cut. This geometry allows the needle to cut for a longer length during insertion before it reaches the full wall thickness of the material at the base of the V cut, which allows for a smoother cutting needle and may promote the gathering of a more complete core than other needles. The 3-D curve could not be ground or cut with EDM using known techniques, but it is attainable with ultrafast CNC laser cutting so long as multiple axis or 3D ablation techniques are used.

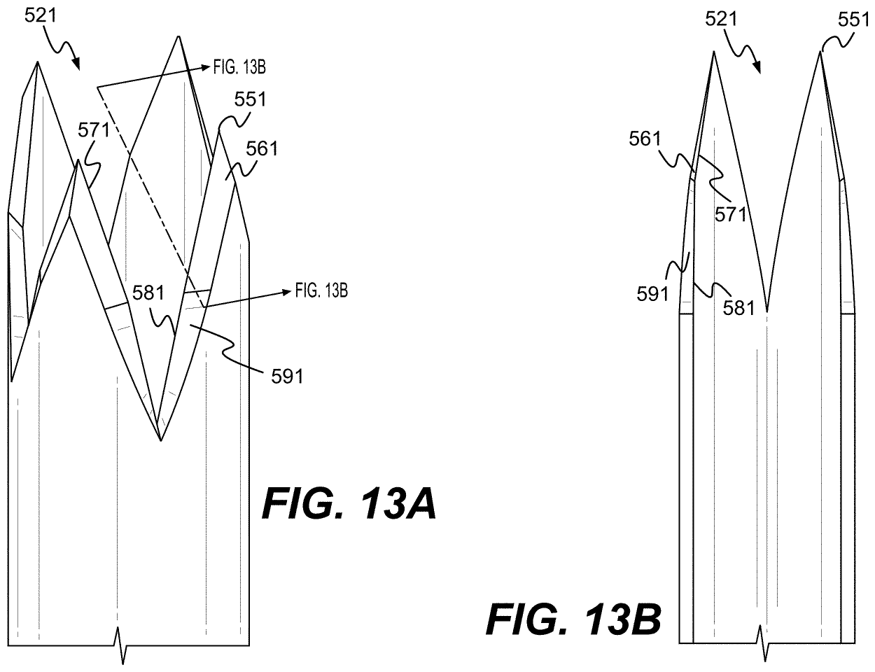

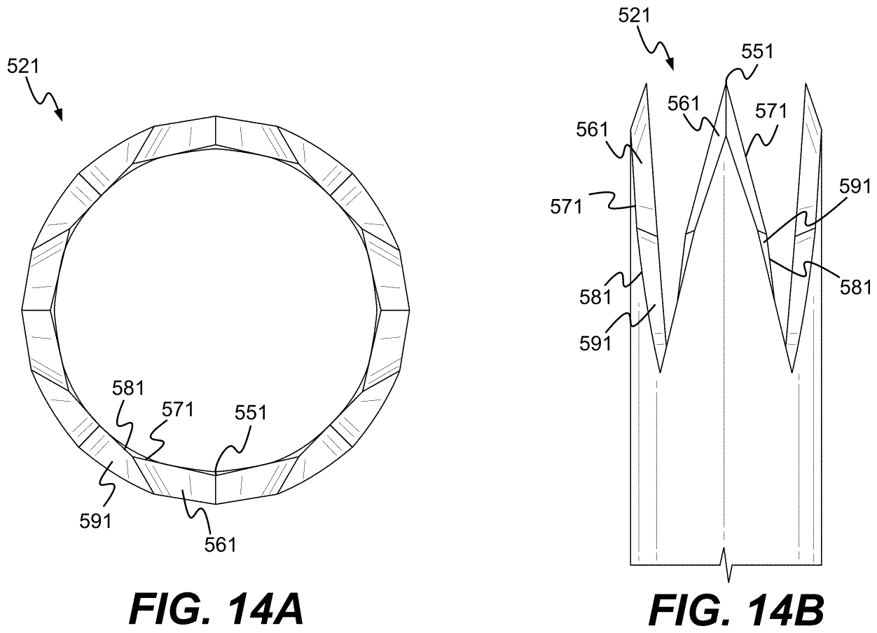

Each tine of needle 520 has one continuous cutting surface from tip to base on each side. In some other embodiments of needle 520, the corkscrew geometry does not end at the base of the V-cut but instead stops at any desired point along the surface between the bevel and the base of the V. Such geometry may prove to be easier to manufacture and/or more effective at tissue cutting. FIGS. 13A, 13B, 14A, and 14B show different views of an embodiment 501 of biopsy needle 500 (including distal end 521, tips 551, cutting surfaces 561, and cutting edges 571) in which the tines are formed by intersections of flat cutting surfaces 561 (which may be ground), and the cutout sections are formed by curved cutting surfaces 591 which corkscrew slowly until reaching the vertical point at the base of the “V” cut. In one example, at least part of the longitudinal extent of curved cutting surface 591 is a ruled surface, such that lines that cross and are tangent to this portion of cutting surface 591 lie within planes orthogonal to the longitudinal axis of the needle. Needle 500 or 501 may be made from hypodermic tubing made of, for example, stainless steel, a cobalt-chromium alloy, or Nitinol (nickel titanium alloy).

It may be desirable to implement needle 200, 201, 202, 203, 204, 205, 300, 400, 401, 500, or 501 to have tines that are equally spaced around the circumference of the needle, with each tine of the needle being symmetric with respect to a plane through the tip of the tine and the longitudinal axis of the needle, and each tine of the needle being identical to each another tine. Rotational and X, Y symmetry can both be important to cutting performance. Symmetry allows the needle to balance the cutting forces between the exterior and interior of the needle, creating a “coring” action. Reducing cutting forces evenly around the circumference increases the force require to push tissue around the needle and reduces the force that allows tissue to enter the needle. Enabling more tissue to enter the needle will result in more tissue being collected by the needle.

Symmetry enables the needle to remain “on course” due to the balance of forces and not veer in one direction, as may occur with asymmetric needles (due to an imbalance of forces). As such, an even number of tines (e.g., 2, 4, 6, etc.) may be preferable to an odd number of tines (e.g., 3, 5, 7, etc.). As compared to biopsy needles having two tines or three tines, a biopsy needle having four or a higher, even number of tines may have a higher rate of success in collecting adequate tissue samples.

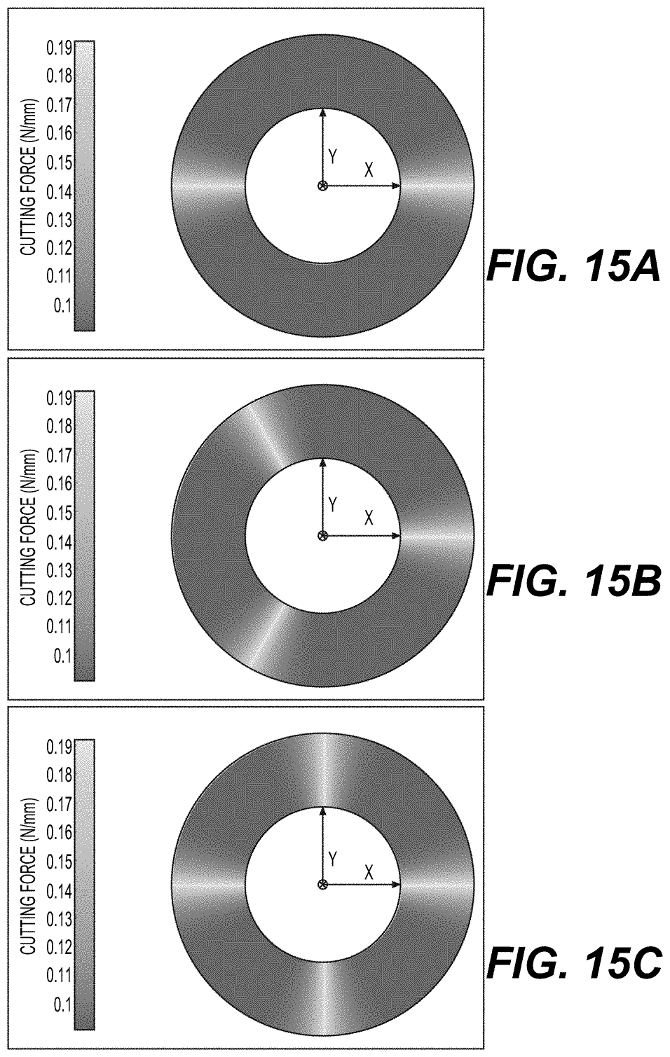

FIG. 15A is a graphical illustration for an exemplary distribution of cutting force along distal end 224 of two-plane biopsy needle 204 of FIG. 6A. FIG. 15B is a graphical illustration for an exemplary distribution of cutting force along distal end 223 of three-plane biopsy needle 205 of FIG. 6B. FIG. 15C is a graphical illustration for an exemplary distribution of cutting force along distal end 220 of the four-plane biopsy needle 200 of FIGS. 4A and 4B.

As shown in FIGS. 15A-15C, during needle insertion, cutting force applied to the tissue being cut is at the lowest at the tine tip and increases with decreasing inclination angle along the cutting edge. Two-plane biopsy needle 204 of FIG. 6A is symmetric over a single longitudinal plane but not over the orthogonal longitudinal plane. Thus, as shown in FIG. 15A, cutting force applied to the tissue being cut by two-plane biopsy needle 204 is only balanced about one longitudinal plane of the needle. Three-plane biopsy needle 205 of FIG. 6B is rotationally symmetric but is not symmetric over a longitudinal plane. Thus, as shown in FIG. 15B, cutting force applied to the tissue being cut by three-plane biopsy needle 205 is only rotationally symmetric, not balanced about any longitudinal plane of biopsy needle 205. The lack of balance of cutting force along two longitudinal planes may allow the tissue being cut to be pushed away or around biopsy needle 205, which may result in loss of tissue cut by distal end 225.

As described herein, one or more parameters of biopsy needle 100 may affect the amount of cutting force applied to the tissue being cut by distal end 120, such as the material, gauge, thickness, number of tines of biopsy needle 100. The values of cutting force of biopsy needle 100 as shown in FIGS. 15A-15C are exemplary.

Advantageously, a four-plane biopsy needle as described herein (e.g., a four-plane implementation of needle 200, 201, 202, 203, 300, 400, 401, 500, or 501) may be implemented to have both rotational symmetry and plane symmetry about two orthogonal longitudinal planes of the needle. The combination of both rotational and plane symmetry allows substantially balanced cutting force to be applied to the tissue by the distal end of the biopsy needle during needle insertion. The substantially balanced cutting force allows the tissue being cut to move towards the center of the biopsy needle and to enter the lumen of the biopsy needle. Otherwise, the tissue would have been pushed away from or pushed around the needle by unbalanced cutting force, as for the two-plane and three-plane biopsy needles. Thus, the rotational symmetric and plane symmetric arrangement of such a four-plane biopsy needle advantageously reduces loss of tissue and/or increases the amount and/or length of the collected tissue sample, thereby increasing the success rate of collecting an adequate tissue sample. The rotational symmetric and plane symmetric arrangement of such a four-plane biopsy needle may further reduce the inadvertent damage to the surrounding tissue at the sampling location.

Additionally, the substantially balanced cutting force applied to the tissue being cut in turn results in substantially balanced reactive force of the tissue applied to the distal end of the needle. The substantially balanced reactive force prevents a four-plane biopsy needle from being veered off course, changing direction, and/or curving in undesired directions during tissue sample collection. Thus, the rotational symmetric and/or plane symmetric arrangement of a four-plane biopsy needle advantageously allows the biopsy needle to maintain a straight sampling path, thereby increasing the accuracy of sampling a desired lesion location.

According to an aspect of the present disclosure, the tines of the biopsy needle are evenly radially spaced apart in rotational symmetry. The tines may be further arranged in plane symmetry about two longitudinal planes that are orthogonal to each other and parallel to the longitudinal axis of the biopsy needle. The rotational symmetric and/or plane symmetric arrangement of the tines of the biopsy needle allows substantially balanced cutting force to be applied to the tissue being cut by the distal end of the biopsy needle during needle insertion. The substantially balanced cutting force allows the tissue being cut, which would be pushed away from or pushed around the needle by unbalanced forces, to move towards the center of the biopsy needle and to enter the lumen of the biopsy needle. Thus, the rotational symmetric and/or plane symmetric arrangement of the tines advantageously increases the amount and/or length of the collected tissue sample, thereby increasing the success rate of collecting an adequate tissue sample.

Additionally, the substantially balanced cutting force applied to the tissue being cut by the distal end of the biopsy needle in turn results in substantially balanced reactive force of the tissue applied to the distal end. The substantially balanced reactive force prevents the biopsy needle from being veered off course, changing direction, and/or curving in undesired directions. Thus, the rotational symmetric and/or plane symmetric arrangement of the tines advantageously allows the biopsy needle to maintain integrity and/or a straight sampling path during needle insertion, thereby increasing the accuracy of sampling a desired lesion location.

As noted above, this disclosure includes descriptions of novel needle geometry that cannot be manufacturing using traditional needle grinding methods. Such needle geometry can only be created using multiple axis ultrafast laser cutting (for example, femtosecond lasers) or other multiple axis techniques. An “ultrafast” laser may be defined as a pulsed laser such that the duration of each pulse is less than one picosecond (e.g., on the order of one femtosecond, ten femtoseconds, or one hundred femtoseconds, such as in the range of one to five hundred femtoseconds, or in the range of ten to three hundred femtoseconds). The duration of each pulse of a fiber laser, in contrast, is on the order of one millisecond. The instantaneous power of a femtosecond laser is also much higher than the instantaneous power of a fiber laser. Femtosecond and other ultrafast lasers can be used to perform so called “cold cutting”: the cutting of a heat-sensitive metal (such as Nitinol) while having a very small heat-affected zone. Non-ultrafast lasers cannot effectively make such cuts due to the larger heat-affected zone (buildup of energy in the material) creating brittleness in the Nitinol. Ultrafast lasers are ideally suited to perform the required precision cutting necessary for these novel needle geometries.

Some of the needle embodiments disclosed herein cannot be made with grinding or even EDM (electrical discharge machining) due to the complex angled geometries. For example, EDM typically requires that the machining wire can extend linearly through the workpiece without intersecting any portions that are not to be machined. Needles having these particular geometries can only be cut using methods with multiple-axis capabilities that can selectively address a single surface of the tube at one time (high speed laser cutting, etc.). Fabrication of the needle tip is completed in a single operation of the CNC cutting equipment, such that the tolerances of the cuts are extremely tight and the geometry is highly repeatable needle-to-needle. In one example, the diameter of the cutting beam of the ultrafast laser is one-tenth of a millimeter at the cutting surface.

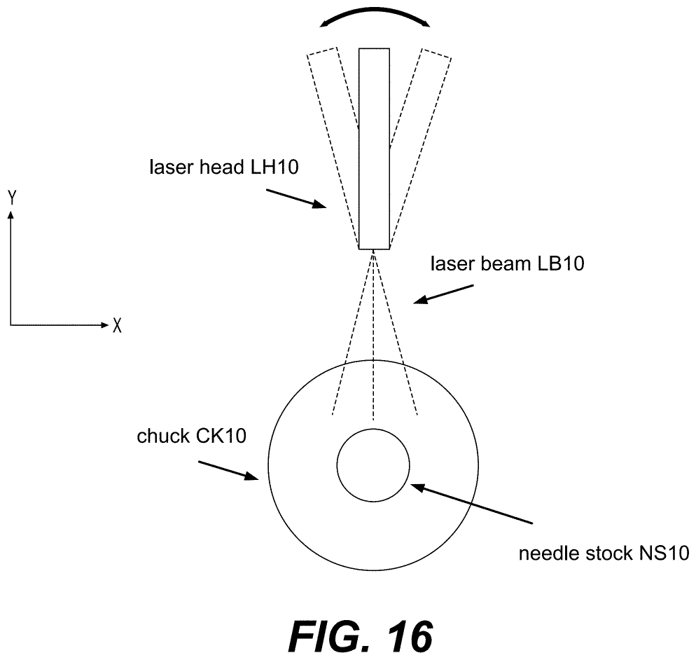

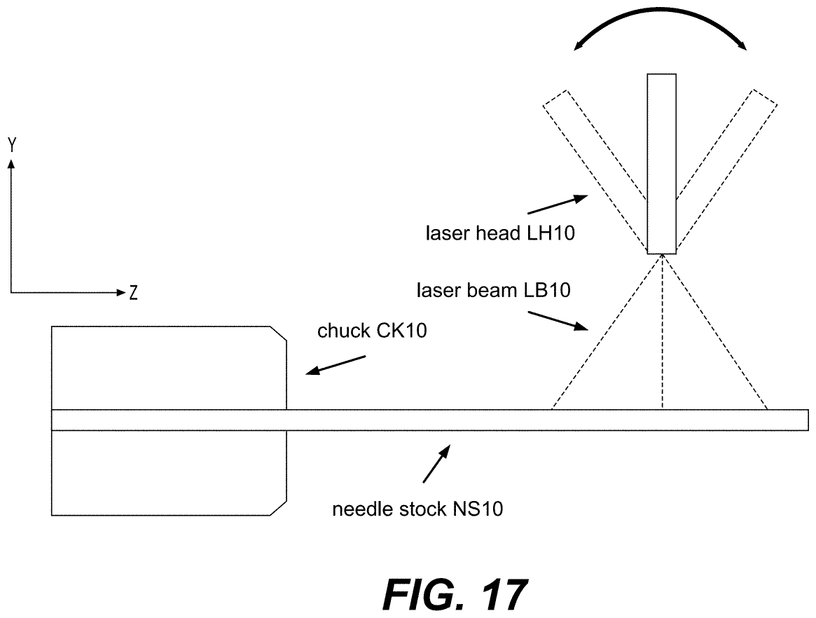

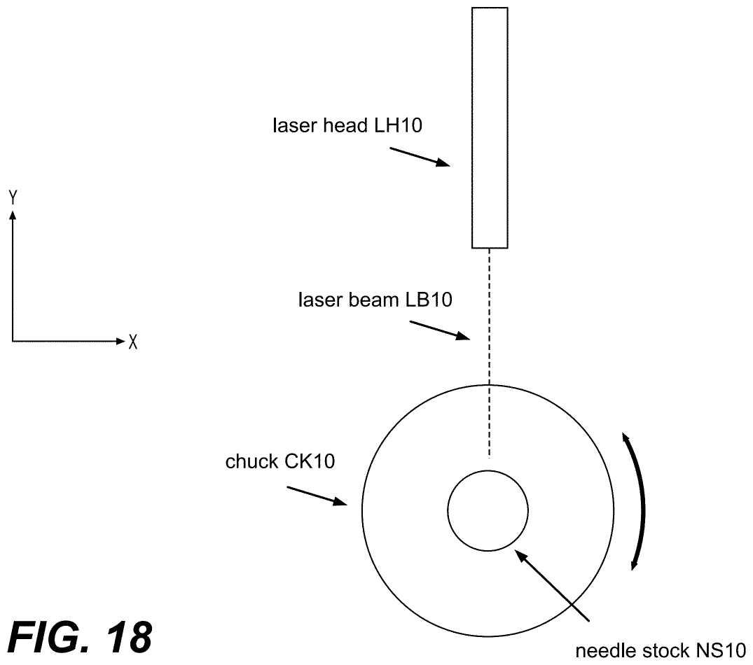

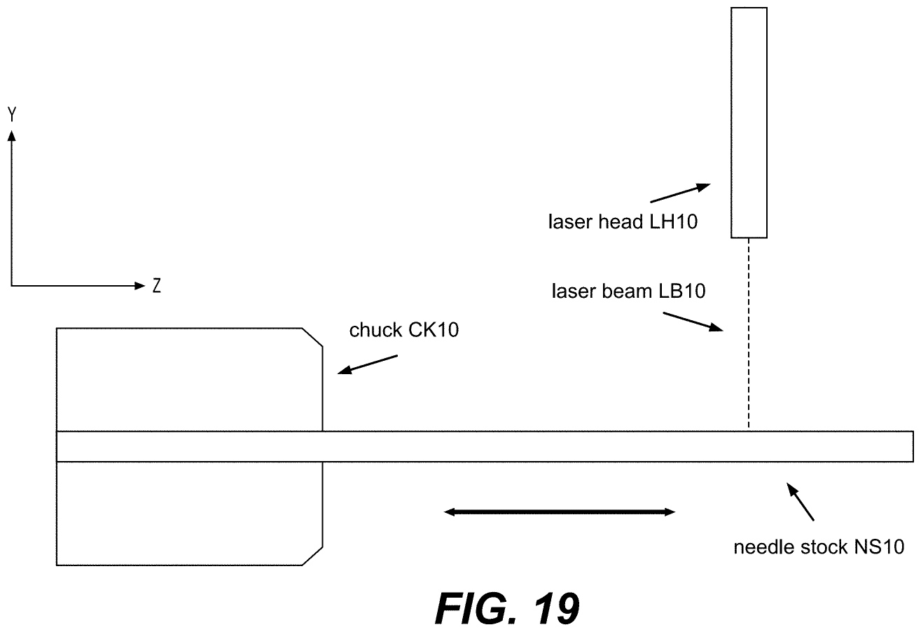

To produce such geometries, it is desirable for the laser head to have the ability to address the hypodermic tubing (typically stainless steel, a cobalt-chromium alloy, or Nitinol (nickel titanium alloy)) from multiple angles and from multiple axes. This can be achieved either by moving the laser head, by moving the tubing itself or by directing the laser beam path through a galvanometer scanner (galvo) system (or similar). In either configuration, it may be desirable for the system to have the freedom to address the tubing radially, typically by rotating the tube. The system may be able to address the tube longitudinally (z direction), typically by moving the tube forward and back but can also be addressed via moving the laser head or directing the beam path. The system may also be able to move across the tube in the x direction and adjust the focal depth of the laser in they direction. The system may also be able to address the tube from angles shallower than 90 degrees, either by tipping the laser head, redirecting the laser beam or tipping the tube. Alternately, a 3D ablation technique could possibly be used to selectively modify ablation depth along a cutting path that would simulate an off-axis cut. Diagrams showing desired degrees of freedom are shown in FIGS. 16-19, with FIGS. 16 and 17 showing degrees of freedom of the laser (e.g., a femtosecond laser) and FIGS. 18 and 19 showing degrees of freedom of the material (e.g., needle stock NS10). FIG. 16 shows an example of relative movement of a cutting spot with respect to a section of needle stock to change an angle of a projection of the cutting beam in a plane orthogonal to the longitudinal axis of the section of needle stock. FIG. 17 shows an example of relative movement of a cutting spot with respect to a section of needle stock to change an angle of a projection of the cutting beam in a plane parallel to the longitudinal axis of the section of needle stock. FIG. 18 shows an example of relative movement of a cutting spot with respect to a section of needle stock to change an angle, in a plane orthogonal to the longitudinal axis of the section of needle stock, of a line that includes the cutting spot and intersects the longitudinal axis. FIG. 19 shows an example of relative movement of a cutting spot along a section of needle stock in a direction parallel to a longitudinal axis of the section of needle stock.

Another aspect of improving biopsy needle collection performance is to increase visibility of needle and/or tip under ultrasound illumination. In general, it may be desirable for a surgeon or other medical professional to move, in a desired manner, a device that is within the body of a patient and cannot be seen. Laparoscopic and endoscopic surgical procedures, for example, commonly include inserting a device into a small incision or existing orifice of the patient's body such that its operative region (e.g., a cutting portion of the device, such as the tip of a needle) is no longer visible. Examples of such devices include (without limitation) needles, cannulas, catheters, and dilators.

Biopsy collection needles that are used with endoscopic ultrasound typically contain echogenic markings on the distal end of the needle, near the tip. These markings are typically made with EDM (electrical discharge machining), grit blasting or laser marking using a fiber laser or Nd:YAG (neodymium-doped yttrium aluminum garnet) laser.

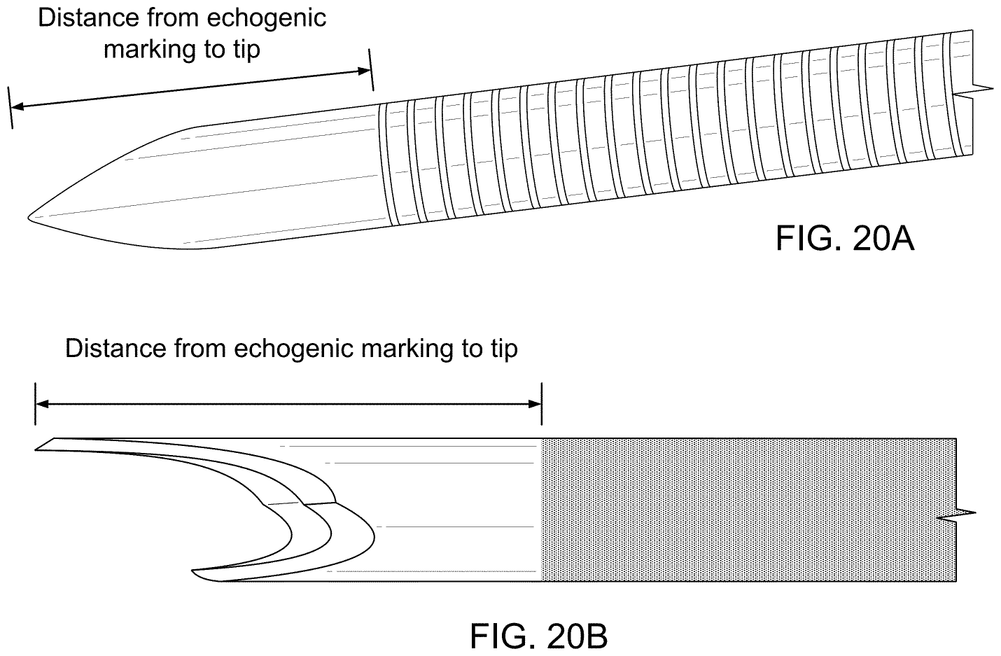

Typically, the needle tip is ground as the first step in the manufacturing process. The echogenic markings are then put on the needle using a separate process. This separate process, whether it be grit blasting, laser marking or EDM, traditionally requires an additional fixture setup to hold the needle and align the needle. The precision of the additional fixturing is limited by the fact that the needle tip cannot be damaged during this step. As such, the tolerancing on the location of the echogenic markings tends err on the side of being too far away from the needle tip rather than too close. The result of this tolerance is that the echogenic markings do not actually mark the needle tip but mark an area a certain distance proximal to the needle tip, usually 3 mm or greater. FIGS. 20A and 20B show this phenomenon as seen in devices currently available.

The offset location of the echogenic markings forces the physician to estimate the location of the needle tip rather than know its exact location. Being forced to estimate the needle tip location is less than optimal for a physician, as the distances between target lesions and blood vessels, nerves or other critical structures may be very small in some situations.

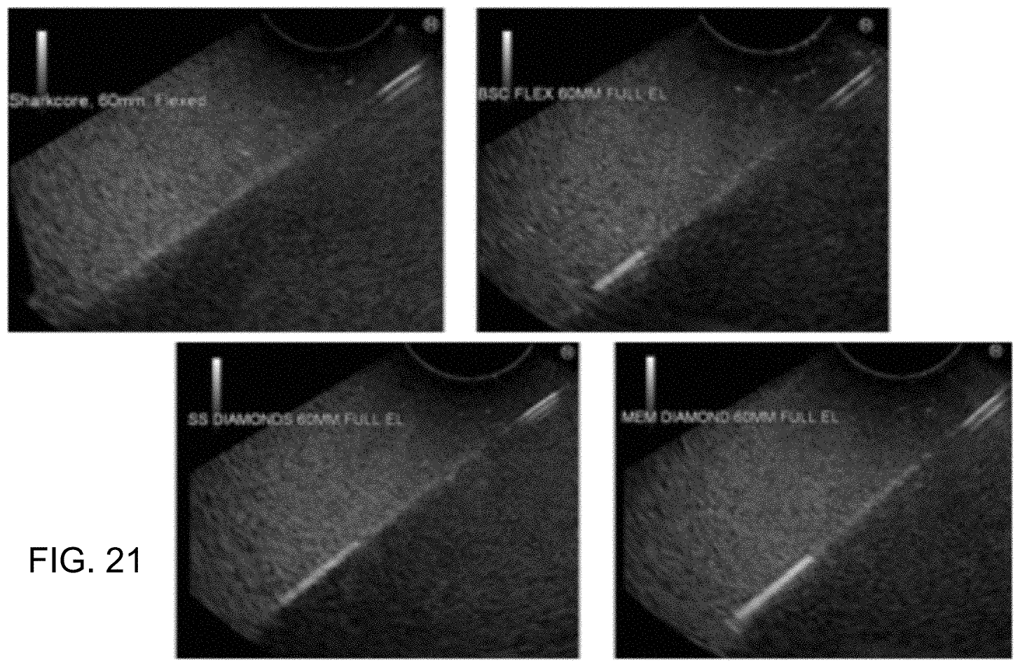

Additionally, echogenic markings made with grit blasting or fiber lasers tend to not be as bright under ultrasound as those made using processes such as EDM and ultrafast lasers. The brighter the needle is under EUS visualization, the more control the physician can have over the needle location. FIG. 21 shows examples of markings made with grit blasting (e.g., using particles of alumina) (top left) and fiber lasers (bottom left) versus markings made with EDM (top right) and an ultrafast laser (bottom right). For grit blasting, the markings are typically not less than five or six millimeters from the needle tip. For EDM, the markings are typically not less than three millimeters from the needle tip.

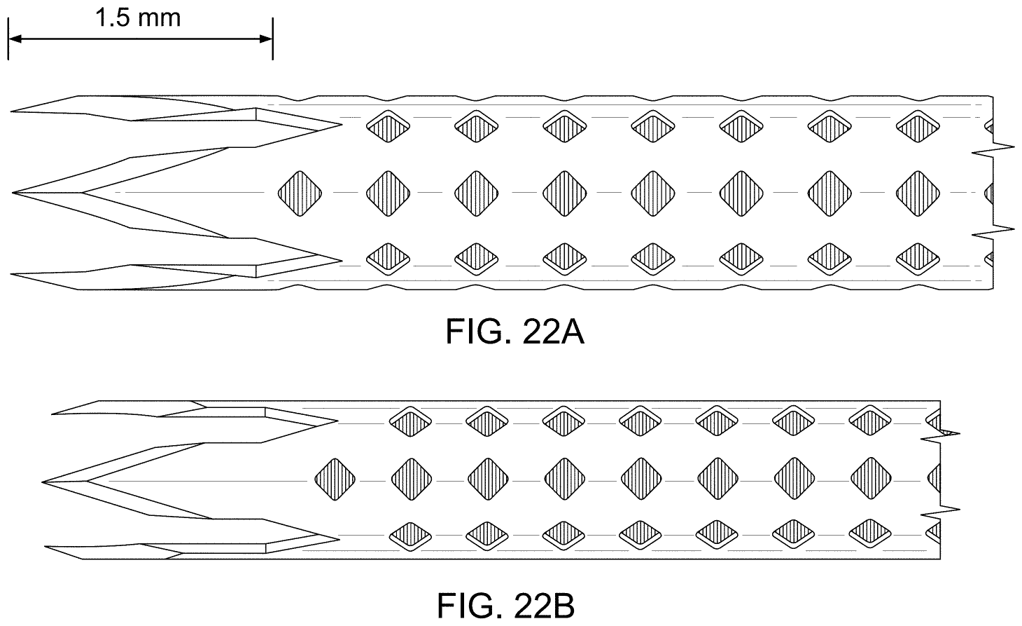

One potential advantage of using ultrafast lasers to create the needle tip is that echogenic markings can be put on the needle during the same operation. The stock tubing can be placed into a single fixture (e.g., a rotating chuck), and the needle tip and the echogenic markings can be made with the same laser without changing fixtures or removing the stock tubing. This continuity enables the tolerances between the laser cut tip and the echogenic marks to be very tight, which can allow the echogenic marks to extend nearly to the very tip of the needle. There is a huge advantage to physicians in knowing more exactly where the needle tip is during EUS visualization. FIG. 22A shows an example of a needle having echogenic markings nearly up to the tip. In this particular example, the distance between the needle tip and the first marking is less than one and one-half times (i.e., less than 150% of) the outside diameter of the needle. The total length of the marked portion of the needle may be in the range of ten to twenty, thirty, fifty, or one hundred millimeters or more. FIG. 22B shows another, similar example.

Due to the process of making the tip and echogenic marks during the same operation, tolerances are excellent and thus echogenic markings can be positioned immediately adjacent to or otherwise very close to the needle tip (e.g., extending within the area of the tine and/or extending within three hundred percent, two hundred percent, one hundred fifty percent, or one hundred thirty percent of the outer diameter of the needle to the needle tip), enabling better tip visualization. A marking may be placed within a tine such that it is entirely closer to the tip of the tine than the base of the tine is, for example, and a marking may even be placed to extend within a bevel plane.

In addition to location precision, making the echogenic markings and cutting the needle tip using the same ultrafast laser operation saves manufacturing time and money, minimizing the number of process steps, reducing part handling, reduces scrap and minimizes secondary cleaning steps (ultrafast laser cutting is extremely clean and produces little slag when compared to grinding).

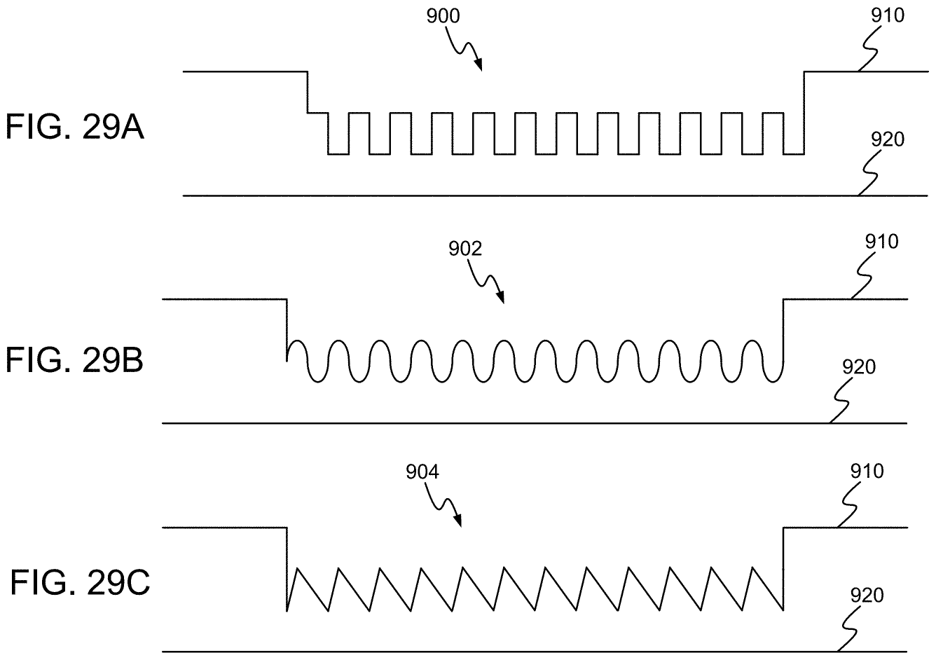

Finally, the use of ultrafast lasers also greatly increases the precision of the echogenic markings themselves, allowing for minute features that can assist with echogenicity. In the example below, not only do the diamond shapes have sharply cut walls and curves but the bottom of the recess is textured with horizontal cuts (e.g., cuts in a plane that is orthogonal to the longitudinal axis of the needle). These cuts effectively transform the bottom into a sine wave of ridges and troughs, improving the scattering of sound and thus improving the brightness of the features under ultrasound visualization. Other textures (e.g., other regular patterns, such as ridges that are steeper on one side than the other as shown e.g. in FIG. 29C) are also possible.

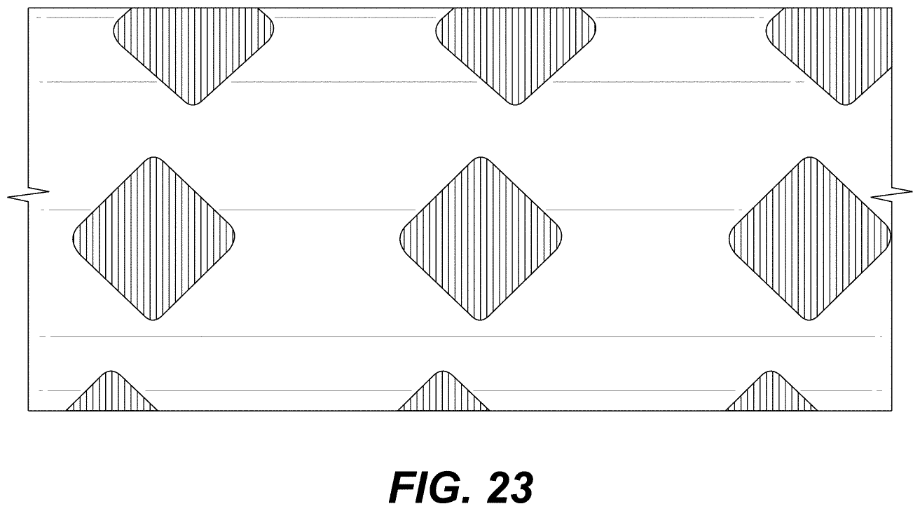

FIG. 23 shows a needle having rows of markings made using an ultrafast laser. In this example, each row extends along the longitudinal axis of the needle, which is from left to right in the figure as shown, and the markings in each row are offset slightly along that axis from corresponding markings in adjacent rows. The bottom surface of the markings in FIG. 23 have a controlled depth (e.g., a depth of one one-thousandth of an inch to the tops of the ridges, and a depth of two one-thousandths of an inch to the bottoms of the ridges, for a needle wall thickness of three one-thousandths of an inch). FIG. 29A-C are cross-sections of examples 900, 902, and 204 of such markings that show the tops and bottom of the ridges (which may be squared, rounded, tilted, etc.) of the regular patterns relative to an outer wall 910 and an inner wall 920 of the needle.

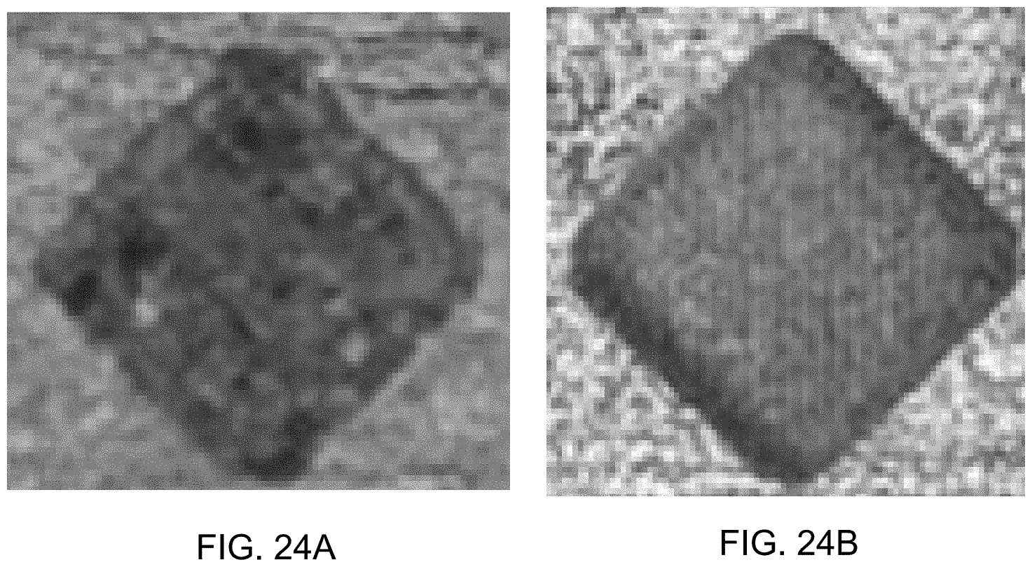

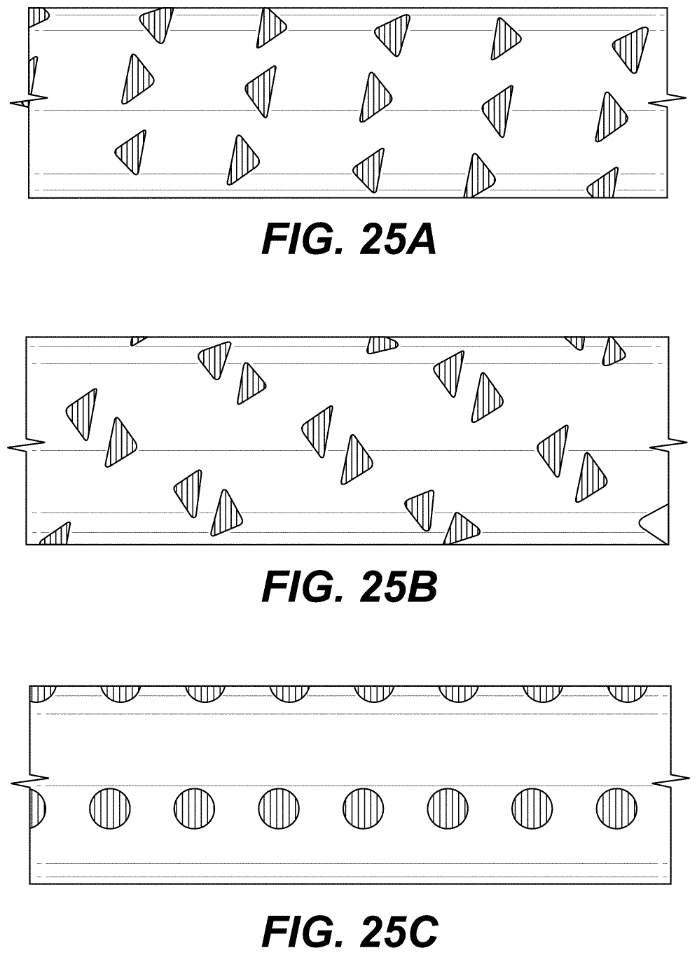

FIG. 24B shows a photograph of one of the markings in FIG. 23, and FIG. 24A shows a photograph of the same marking made using a non-ultrafast fiber laser. In contrast to the regular pattern seen on the bottom surface of the marking in FIG. 24B, the bottom surface of the marking of the needle shown in FIG. 24A is random and formed by dross that was ablated and then re-solidified. While the bottom surface of the marking in FIG. 24B has a controlled depth (e.g., a depth of one one-thousandth of an inch to the tops of the ridges, and a depth of two one-thousandths of an inch to the bottoms of the ridges, for a needle wall thickness of three one-thousandths of an inch), the process used to create the marking in FIG. 24A has effectively no control of depth in comparison.

Other embodiments of this same method include alternate shapes and patterns, as seen e.g. in the examples of 25A-C. It is also possible to use markings that are longer in the direction of the longitudinal axis of the needle than in the orthogonal direction (e.g., from two to five times or more), and even to use markings that are longitudinal stripes (e.g., of length three to five or ten millimeters or more), so long as the structural integrity of the resulting needle is sufficient. It is contemplated and disclosed that rows, columns, or other groupings of features with ridged bottoms may alternate with similar groupings of features without ridged bottoms and/or with similar groupings of features having differently textured bottoms (e.g., being ridged in a direction that is orthogonal to that of the other ridging).

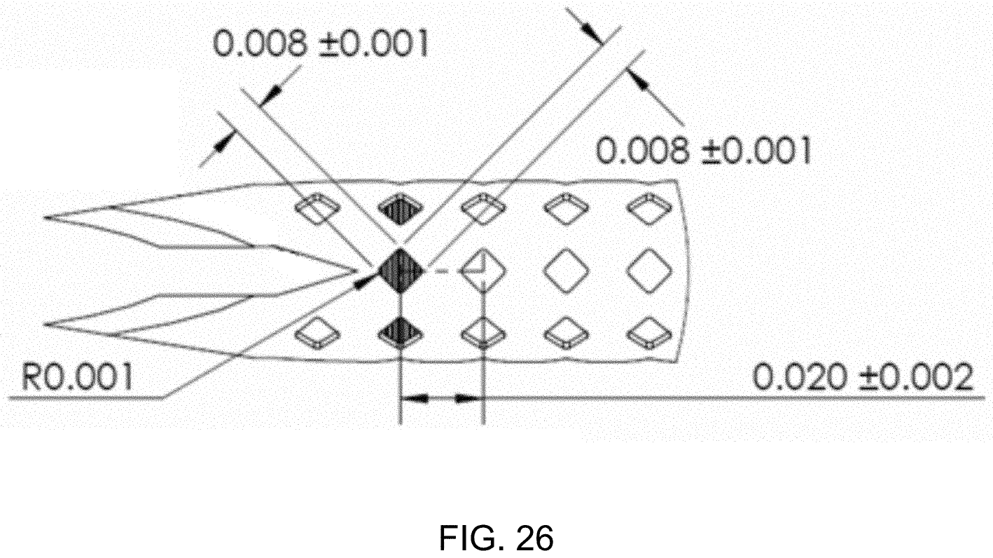

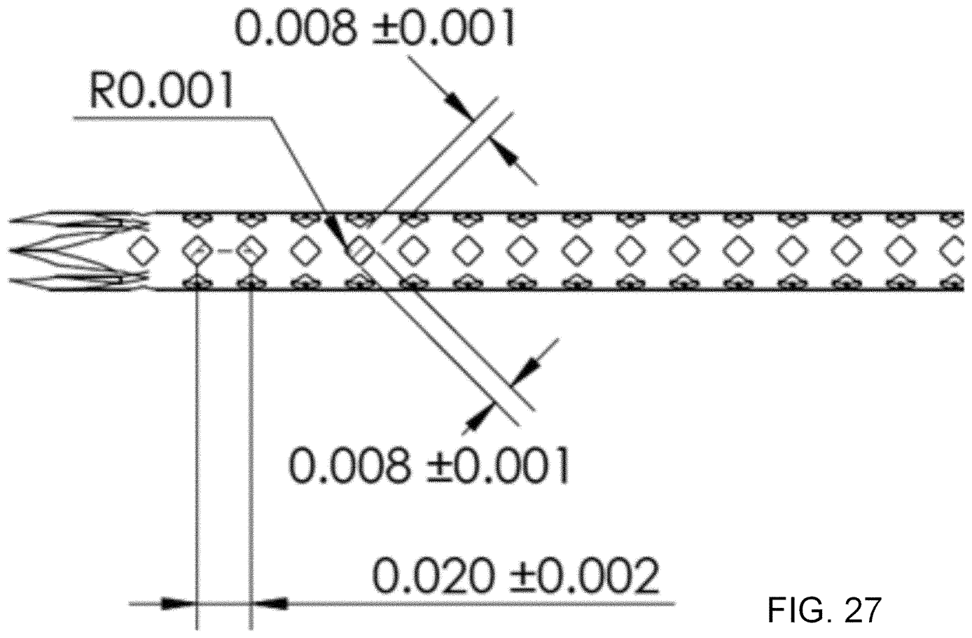

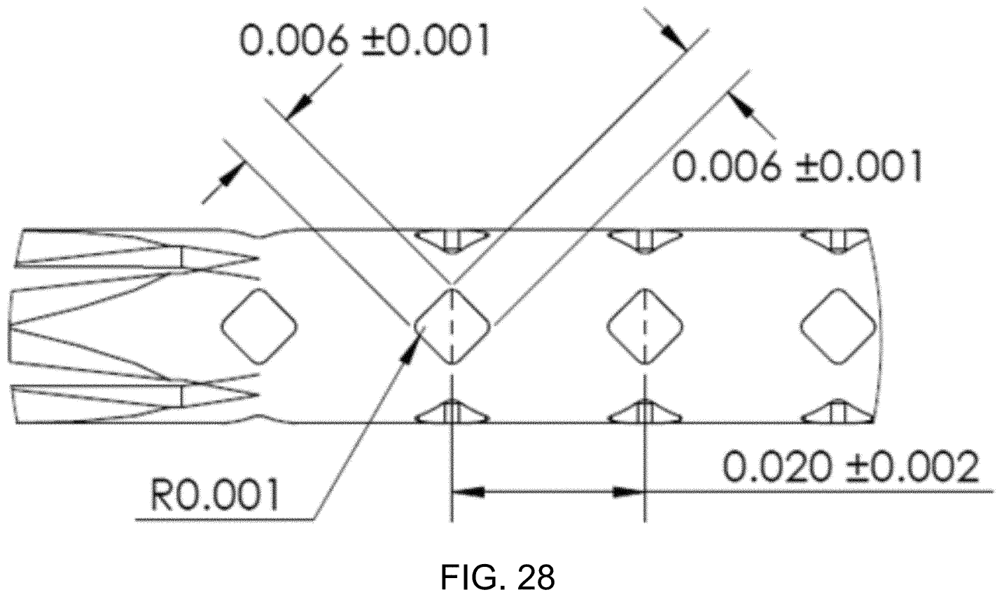

It has been found that the diamonds as shown in FIGS. 23 and 24B have high echogenicity when arranged in the patterns and spacings shown in FIGS. 26-28. In these figures, the indicated dimensions are in inches, and the notation “R0.001” indicates that the corner of the marking has a radius of one one-thousandth of an inch. Such dimensions are appropriate for use with ultrasound frequencies across a range of three to twelve megahertz, and may be scaled proportionally (e.g., with reference to a center frequency of seven or seven-and-one-half megahertz) for a smaller range of frequencies and/or for frequencies outside this range.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A needle comprising:
an elongated body extending along a longitudinal axis, the elongated body comprising:
a lumen extending therethrough, and
a distal end comprising at least two tines,
wherein a heel between adjacent ones of the at least two tines comprises an intersection of two cutting surfaces, and
wherein each of the two cutting surfaces comprises a cutting edge having an inclination angle of at least thirty degrees at each point between a first end of the cutting edge and a second end of the cutting edge, and
wherein, for each of the two cutting surfaces, an angle between the cutting surface and an inner surface of the elongated body is not greater than forty-five degrees in a plane that includes the longitudinal axis.

2. The needle according to claim 1, wherein each of the cutting edges is straight between the first end and the second end.

3. The needle according to claim 1, wherein each of the cutting edges has an average inclination angle of at least fifty degrees between the first end and the second end.

4. The needle according to claim 1, wherein each of the two cutting edges has an inclination angle of at least thirty degrees along at least ninety percent of its entire length.

5. The needle according to claim 1, wherein a tip of each of the at least two tines comprises an intersection of two cutting edges that each have an inclination angle of at least forty-five degrees along its length.

6. The needle according to claim 1, wherein one of the two cutting edges extends continuously to one of the adjacent ones of the at least two tines, and the other of the two cutting edges extends continuously to the other of the adjacent ones of the at least two tines.

7. The needle according to claim 1, wherein one of the two cutting edges extends in a continuous curve to one of the adjacent ones of the at least two tines, and the other of the two cutting edges extends in a continuous curve to the other of the adjacent ones of the at least two tines.

8-10. (canceled)

11. A needle comprising:
an elongated body extending along a longitudinal axis, the elongated body comprising:
a lumen extending therethrough, and
a distal end comprising at least two tines,
wherein a tip of each of the at least two tines is formed by an intersection of two cutting surfaces, each of the cutting surfaces lying in a respective bevel plane, and
wherein at least one of the bevel planes intersects the needle.

12. The needle according to claim 11, wherein one of the cutting surfaces that intersects to form a first one of the at least two tines intersects one of the cutting surfaces that intersects to form a second one of the at least two tines.

13. The needle according to claim 11, wherein each of the cutting surfaces has a cutting edge, and
wherein each of the cutting edges has an average inclination angle of at least fifty degrees.

14. The needle according to claim 11, wherein each of the cutting surfaces has a cutting edge, and wherein each of the cutting edges has an inclination angle of at least thirty degrees along at least ninety percent of its entire length.

15. The needle according to claim 11, wherein the at least two tines comprises four tines.

16. The needle according to claim 11, wherein a direction of each of the at least one bevel planes is not more than sixty degrees away from a tip formed by the corresponding cutting surface.

17. The needle according to claim 11, wherein, for each of the tips of each of the at least two tines, an inclination angle of a cutting edge between the tip and an outer wall of the elongated body is at least thirty degrees along at least ninety percent of its entire length.

18-30. (canceled)

31. A needle comprising:
an elongated body extending along a longitudinal axis, the elongated body comprising:
a lumen extending therethrough, and
a distal end comprising at least two tines,
wherein each of the at least two tines includes at least one curved cutting surface, and
wherein, for the at least one curved cutting surface of each tine, a first line that crosses and is tangent to the curved cutting surface has a first direction, and a second line that crosses and is tangent to the curved cutting surface has a second direction different from the first direction.

32. The needle according to claim 31, wherein a tip of each of the at least two tines is formed by an intersection of two cutting surfaces, each of the cutting surfaces lying in a respective bevel plane.

33. The needle according to claim 31, wherein a tip of each of the at least two tines comprises an intersection of two cutting edges that each have an inclination angle of at least forty-five degrees along its length.

34. The needle according to claim 31, wherein each of the curved cutting surfaces has a cutting edge with an average inclination angle of at least fifty degrees.

35. The needle according to claim 31, wherein each of the curved cutting surfaces has a cutting edge with an inclination angle of at least thirty degrees along at least ninety percent of its entire length.

36. The needle according to claim 31, wherein the at least two tines comprises four tines.

37. The needle according to claim 31, wherein each of the curved cutting surfaces extends continuously from a tip of a corresponding one of the at least two tines to an adjacent one of the curved cutting surfaces.

38-50. (canceled)
